SEQUENCE LISTING (9 sequences)

SEQ ID NO: 1 - DNA, 1116 bp, Artificial
  synthetic codon-optimized k-carrageenase
gctagcatgcaaccacctatcgctaaaccaggagaaacatggattcttcaagcaaaacgttctgatgaatttaacgttaaagacgctactaaatggaacttccaaacagaaaactatggtgtatggtcttggaaaaacgaaaatgcaactgtttcaaacggtaaacttaaattaactacaaaacgtgaatctcaccaaagaacattctgggatggttgcaaccaacaacaagttgcaaactacccactttattacacttctggtgttgcaaaatcacgtgctacaggaaactacggttattacgaagcacgtatcaaaggagcatctactttccctggtgtatctccagctttctggatgtactctacaattgaccgtagccttactaaagaaggtgacgttcaatactctgaaatcgacgtagttgaacttacacaaaaatcagcagttcgtgaatctgaccacgatcttcacaacattgtagttaaaaacggtaaacctacatggatgcgcccgggttcttttcctcaaactaaccataacggctaccaccttccatttgatcctcgtaacgacttccacacatacggagttaacgtaactaaagataaaatcacatggtatgttgacggtgaaattgtaggagaaaaagacaacctttattggcaccgtcaaatgaacttaactctttctcaaggccttagagcgcctcacacacaatggaaatgcaaccaattctacccatcagcaaacaaatctgctgaaggtttccctacttcaatggaagtagactacgttcgtacatgggttaaagtaggaaacaacaattctgcaccaggtgaaggacaatcatgtcctaacacattcgttgctgtaaactctgttcaactttcagctgcaaaacaaactcttcgtaaaggtcaatctacaactttagaatcaactgttcttccaaactgcgcaacaaacaaaaaagttatctactctagctcaaacaaaaacgtagctactgttaactctgcaggtgttgtaaaagcaaaaaacaaaggtacagctactattacagttaaaacaaaaaacaaaggaaaaatcgataaacttacaatcgcagtaaac

SEQ ID NO: 2 - protein, 372 aa, Artificial
  synthetic k-carrageenase
ASMQPPIAKPGETWILQAKRSDEFNVKDATKWNFQTENYGVWSWKNENATVSNGKLKLTTKRESHQRTFWDGCNQQQVANYPLYYTSGVAKSRATGNYGYYEARIKGASTFPGVSPAFWMYSTIDRSLTKEGDVQYSEIDVVELTQKSAVRESDHDLHNIVVKNGKPTWMRPGSFPQTNHNGYHLPFDPRNDFHTYGVNVTKDKITWYVDGEIVGEKDNLYWHRQMNLTLSQGLRAPHTQWKCNQFYPSANKSAEGFPTSMEVDYVRTWVKVGNNNSAPGEGQSCPNTFVAVNSVQLSAAKQTLRKGQSTTLESTVLPNCATNKKVIYSSSNKNVATVNSAGVVKAKNKGTATITVKTKNKGKIDKLTIAVN

SEQ ID NO: 3 - DNA, 828 bp, Artificial
  synthetic codon-optimized k-carrageenase
gctagcatgcaaccacctatcgctaaaccaggagaaacatggattcttcaagcaaaacgttctgatgaatttaacgttaaagacgctactaaatggaacttccaaacagaaaactatggtgtatggtcttggaaaaacgaaaatgcaactgtttcaaacggtaaacttaaattaactacaaaacgtgaatctcaccaaagaacattctgggatggttgcaaccaacaacaagttgcaaactacccactttattacacttctggtgttgcaaaatcacgtgctacaggaaactacggttattacgaagcacgtatcaaaggagcatctactttccctggtgtatctccagctttctggatgtactctacaattgaccgtagccttactaaagaaggtgacgttcaatactctgaaatcgacgtagttgaacttacacaaaaatcagcagttcgtgaatctgaccacgatcttcacaacattgtagttaaaaacggtaaacctacatggatgcgcccgggttcttttcctcaaactaaccataacggctaccaccttccatttgatcctcgtaacgacttccacacatacggagttaacgtaactaaagataaaatcacatggtatgttgacggtgaaattgtaggagaaaaagacaacctttattggcaccgtcaaatgaacttaactctttctcaaggccttagagcgcctcacacacaatggaaatgcaaccaattctacccatcagcaaacaaatctgctgaaggtttccctacttcaatggaagtagactacgttcgtacatgggttaaagtaggaaacaacaat

SEQ ID NO: 4 - protein, 276 aa, Artificial
  synthetic k-carrageenase
ASMQPPIAKPGETWILQAKRSDEFNVKDATKWNFQTENYGVWSWKNENATVSNGKLKLTTKRESHQRTFWDGCNQQQVANYPLYYTSGVAKSRATGNYGYYEARIKGASTFPGVSPAFWMYSTIDRSLTKEGDVQYSEIDVVELTQKSAVRESDHDLHNIVVKNGKPTWMRPGSFPQTNHNGYHLPFDPRNDFHTYGVNVTKDKITWYVDGEIVGEKDNLYWHRQMNLTLSQGLRAPHTQWKCNQFYPSANKSAEGFPTSMEVDYVRTWVKVGNNN

SEQ ID NO: 5 - protein, 96 aa, Artificial
  synthetic k-carrageenase
SAPGEGQSCPNTFVAVNSVQLSAAKQTLRKGQSTTLESTVLPNCATNKKVIYSSSNKNVATVNSAGVVKAKNKGTATITVKTKNKGKIDKLTIAVN

SEQ ID NO: 6 - protein, 397 aa, Alteromonas carrageenovora
MKPISIVAFPIPAISMLLLSAVSQAASMQPPIAKPGETWILQAKRSDEFNVKDATKWNFQTENYGVWSWKNENATVSNGKLKLTTKRESHQRTFWDGCNQQQVANYPLYYTSGVAKSRATGNYGYYEARIKGASTFPGVSPAFWMYSTIDRSLTKEGDVQYSEIDVVELTQKSAVRESDHDLHNIVVKNGKPTWMRPGSFPQTNHNGYHLPFDPRNDFHTYGVNVTKDKITWYVDGEIVGEKDNLYWHRQMNLTLSQGLRAPHTQWKCNQFYPSANKSAEGFPTSMEVDYVRTWVKVGNNNSAPGEGQSCPNTFVAVNSVQLSAAKQTLRKGQSTTLESTVLPNCATNKKVIYSSSNKNVATVNSAGVVKAKNKGTATITVKTKNKGKIDKLTIAVN

SEQ ID NO: 7 - DNA, 1136 bp, Artificial
  synthetic codon-optimized k-carrageenase
gcgcgcaggcagctagcatgcaaccacctatcgctaaaccaggagaaacatggattcttcaagcaaaacgttctgatgaatttaacgttaaagacgctactaaatggaacttccaaacagaaaactatggtgtatggtcttggaaaaacgaaaatgcaactgtttcaaacggtaaacttaaattaactacaaaacgtgaatctcaccaaagaacattctgggatggttgcaaccaacaacaagttgcaaactacccactttattacacttctggtgttgcaaaatcacgtgctacaggaaactacggttattacgaagcacgtatcaaaggagcatctactttccctggtgtatctccagctttctggatgtactctacaattgaccgtagccttactaaagaaggtgacgttcaatactctgaaatcgacgtagttgaacttacacaaaaatcagcagttcgtgaatctgaccacgatcttcacaacattgtagttaaaaacggtaaacctacatggatgcgcccgggttcttttcctcaaactaaccataacggctaccaccttccatttgatcctcgtaacgacttccacacatacggagttaacgtaactaaagataaaatcacatggtatgttgacggtgaaattgtaggagaaaaagacaacctttattggcaccgtcaaatgaacttaactctttctcaaggccttagagcgcctcacacacaatggaaatgcaaccaattctacccatcagcaaacaaatctgctgaaggtttccctacttcaatggaagtagactacgttcgtacatgggttaaagtaggaaacaacaattctgcaccaggtgaaggacaatcatgtcctaacacattcgttgctgtaaactctgttcaactttcagctgcaaaacaaactcttcgtaaaggtcaatctacaactttagaatcaactgttcttccaaactgcgcaacaaacaaaaaagttatctactctagctcaaacaaaaacgtagctactgttaactctgcaggtgttgtaaaagcaaaaaacaaaggtacagctactattacagttaaaacaaaaaacaaaggaaaaatcgataaacttacaatcgcagtaaactaaaagctt

SEQ ID NO: 8 - DNA, 11 bp, Artificial
  synthetic signal sequence
gcgcgcaggca

SEQ ID NO: 9 - DNA, 9 bp, Artificial
  synthetic stop sequence
taaaagctt

(19) United States
(12) Patent Application Publication
(10) Pub. No.: US 2009/0048136 A1
(43) Pub. Date: Feb. 19, 2009

(54) KAPPA-CARRAGEENASE AND KAPPA-CARRAGEENASE-CONTAINING COMPOSITIONS

(76) Inventors: Hugh C. McDonald, Carlsbad, CA (US); Brian Schmidt, Palo Alto, CA (US)

Correspondence Address: LYNN MARCUS-WYNER;Danisco US, Inc., Genencor Division, 925 PAGE MILL ROAD, PALO ALTO, CA, 94304-1013, US

(21) Appl. No.: 11/838,884

(22) Filed: Aug. 15, 2007

(51) Int. Cl. C11D 7/42; C12N 15/11; C12N 15/00; D06L 1/04; C11D 3/16; C12S 11/00; C12S 9/00; C12N 1/20; C12P 21/04

(52) U.S. Cl. 510/281; 536/23.2; 435/320.1; 435/252.3; 435/252.31; 435/69.1; 510/392; 510/320

(57) ABSTRACT

The present invention provides cleaning compositions comprising at least one carrageenase enzyme, methods for producing carrageenase enzymes in host cells, host cells comprising recombinant polynucleotides encoding at least one carrageenase, and recombinant polynucleotides encoding carrageenase.

### FIELD OF THE INVENTION

The present invention provides cleaning compositions comprising at least one carrageenase enzyme, methods for producing carrageenase enzymes in host cells, host cells comprising recombinant polynucleotides encoding at least one carrageenase, and recombinant polynucleotides encoding carrageenase.

### BACKGROUND

Detergent and other cleaning compositions typically include a complex combination of active ingredients. For example, most cleaning products include a surfactant system, enzymes for cleaning, bleaching agents, builders, suds suppressors, soil-suspending agents, soil-release agents, optical brighteners, softening agents, dispersants, dye transfer inhibition compounds, abrasives, bactericides, and perfumes. Despite the complexity of current detergents, there are many stains that are difficult to completely remove due to the complexity of stain mixtures, particularly those that include fibrous material, particularly those that comprise carbohydrates and/or carbohydrate derivatives, fiber, cell wall components (e.g., plant material, wood, mud/clay based soil, and fruit), and food additives (e.g. food texturizing and stabilizing additives such as carrageenans). Thus, there remains a need in the art for detergent components that are effective in removing such fibrous materials.

### SUMMARY OF THE INVENTION

The present invention provides cleaning compositions comprising at least one carrageenase enzyme, methods for producing carrageenase enzymes in host cells, host cells comprising recombinant polynucleotides encoding at least one carrageenase, and recombinant polynucleotides encoding carrageenase.

The present invention relates to polynucleotides encoding carrageenase polypeptides, and host cells that have been genetically manipulated to produce carrageenase enzymes. In particular, the present invention relates to Gram-positive microorganisms having exogenous nucleic acid sequences encoding at least one carrageenase introduced therein and methods for producing the carrageenase proteins in such host cells. In some preferred embodiments, the Gram-positive microorganisms are members of the genus Bacillus. In some particularly preferred embodiments, the present invention provides methods and compositions for the production of kappa-carrageenase (“κ-carrageenase”) by a Bacillus host cell.

In some embodiments, the invention provides for an isolated recombinant polynucleotide comprising a sequence encoding a kappa-carrageenase enzyme, wherein the isolated polynucleotide is operably linked to a polynucleotide sequence that encodes a secretory signal peptide. In some embodiments, the heterologous sequence encoding the secretory signal peptide is derived from a Gram-positive microorganism. In some embodiments, the secretory signal peptide is the AprE signal peptide. In some embodiments, the sequence encoding the carrageenase enzyme comprises a sequence that is optimized from a polynucleotide encoding a wild-type carrageenase (e.g., a wild-type kappa-carrageenase obtained from the Alteromonas sp.). In some preferred embodiments, the sequence encoding the carrageenase comprises a sequence that is optimized from a polynucleotide encoding a wild-type carrageenase obtained from Alteromonas carrageenovora. In some embodiments, the optimized polynucleotide sequence is at least about 70% identical to SEQ ID NO:1 or SEQ ID NO:3. In other embodiments, the codon-optimized sequence is at least about 75% identical to SEQ ID NO:1 or SEQ ID NO:3, or more preferably about 80%, about 85%, about 90%, about 95%, about 97%, about 98%, or about 99% identical to SEQ ID NO:1 or SEQ ID NO:3.

In some embodiments, the invention provides for an expression vector that comprises an isolated polynucleotide comprising a sequence encoding a kappa-carrageenase enzyme, wherein the isolated polynucleotide is operably linked to a heterologous sequence that encodes a secretory signal peptide. In some preferred embodiments, the heterologous sequence comprises the AprE signal peptide.

The invention also provides for host cells that comprise the expression vector of the present invention. In some embodiments, the host cell is a Bacillus host cell that comprises a recombinant nucleic acid encoding a fusion protein comprising a signal sequence and a carrageenase protein, wherein the host cell secretes kappa-carrageenase protein from the cell, and wherein the host cell has inactivated protease genes. In some embodiments, the Bacillus host cell secretes a carrageenase protein that has an amino acid sequence that is at least about 70% identical to SEQ ID NO:4. In some embodiments, the kappa-carrageenase has at least about 75%, still more preferably at least about 80%, more preferably about 85%, yet more preferably about 90%, even more preferably at least about 95%, and most preferably about 99% identity with SEQ ID NO:4.

In some preferred embodiments, the host cells have inactivated nprE, aprE, epr, ispA, and/or bpr protease genes. In some other embodiments, the host cells have inactivated nprE and aprE protease genes (See e.g., U.S. Pat. No. 5,387,521, incorporated by reference in its entirety). In other embodiments, the host cell comprises a signal sequence that is encoded by the aprE gene of B. subtilis. The recombinant polynucleotide contained in the host cell is in some embodiments operably linked to a promoter and terminator to form an expression cassette. In some other embodiments, the recombinant nucleic acid is present in the genome of the host cell, while in other embodiments, it is present in a vector that autonomously replicates in the host cell. In some preferred embodiments, the recombinant nucleic acid is codon optimized for expressing the carrageenase fusion protein in the Bacillus host cell.

A culture of cells comprising a plurality of the above-described Bacillus host cells and culture medium is also provided. The culture of cells in preferred embodiments comprises the kappa-carrageenase protein. In some embodiments, the cells in the culture comprise the kappa-carrageenase protein. In other embodiments, the culture medium comprises the kappa-carrageenase protein. In yet other embodiments, the culture cells and the culture medium comprise the kappa-carrageenase protein.

The present invention also provides for methods of producing a kappa-carrageenase protein, comprising culturing the above-described Bacillus host cell to provide under conditions such that the expressed kappa-carrageenase protein is secreted into the culture medium used to culture the Bacillus host cell. In some preferred embodiments, the methods may further comprise the step of recovering the kappa-carrageenase protein from said culture medium. The use of any suitable recovery method is contemplated to find use in the methods of the present invention.

The present invention also provides cleaning compositions comprising carrageenase activity. In some preferred embodiments, the cleaning compositions comprise at least one kappa-carrageenase, comprising an amino acid sequence that is at least about 70% identical to the kappa-carrageenase set forth in SEQ ID NO:4. In some embodiments, the kappa-carrageenase has at least about 75%, still more preferably at least about 80%, more preferably about 85%, yet more preferably about 90%, even more preferably at least about 95%, and most preferably about 99% identity with SEQ ID NO:4. In some embodiments, the cleaning composition is a detergent. In some embodiments, these detergent cleaning compositions further include other enzymes (e.g., proteases, amylases, mannanases, peroxidases, oxido reductases, cellulases, lipases, cutinases, pectinases, pectin lyases, xylanases, and/or endoglycosidases), as well as builders and stabilizers.

The present invention also provides methods of cleaning, the comprising contacting a surface and/or an article (e.g., material, such as fabric) with the cleaning composition of the present invention; and optionally washing and/or rinsing the surface or article. In some alternative embodiments, any suitable composition provided herein finds use in cleaning methods. In some embodiments, the fabric comprises at least one stain comprising a kappa- and/or a lambda-carrageenan. In some particularly preferred embodiments, the cleaning compositions of the present invention find use in removing salad dressing, barbeque sauce, and/or marmalade from fabrics.

### BRIEF DESCRIPTION OF THE DRAWINGS

FIG. 1 shows the amino acid sequence of wild-type CgkA k-Carrageenase from Alteromonas carrageenovora (Potin et al., Eur. J. Biochem. 228:971-5 (1995)). The native signal sequence is underlined; the mature coding portion is in bold; and the C-terminal pro sequence is in italics.

FIG. 2 shows the polynucleotide sequence of the synthetic codon-optimized gene encoding the mature portion and pro region of κ-carrageenase as cloned into a DNA2.0 vector resulting in vector pJ31-7585. The mature and pro portions of the κ-carrageenase are shown in bold; the mature portion is also underlined. The DNA sequence encoding the end of the AprE signal sequence (GCGCGCAGGCA) and the stop sequence (TAAAAGCTT) are shown in italics.

FIG. 3 show a map of the p2JM-cgkA integration vector containing the codon-optimized k-carrageenase.

FIG. 4 shows bar diagrams depicting the hydrolytic activity of k-carrageenase on three types of carrageenans, and in three types of detergents.

FIGS. 4 A-C show bar diagrams depicting the hydrolytic activity of k-carrageenase in AATCC HDL detergent on kappa- (type I), iota- (type II), and kappa- (type III) carrageenans, respectively. FIGS. 4D and 4E depict the activity of k-carrageenase in HDD and ADW detergents, respectively, on kappa-carrageenan (type I).

FIGS. 5A and 5B show bar diagrams depicting the activity of k-carrageenase in ATCC HDL detergent on salad dressing and barbeque stain, respectively. The soil removing activity was determined using the microswatch method described in Example 3.

FIG. 6 shows a bar diagram depicting the activity of k-carrageenase in ATCC HDL detergent on stains of tomato juice, tikka, grass, marmalade, ketchup, mustard, barbeque sauce and chocolate soy. The soil removing activity was determined using the tergotometer method described in Example 4.

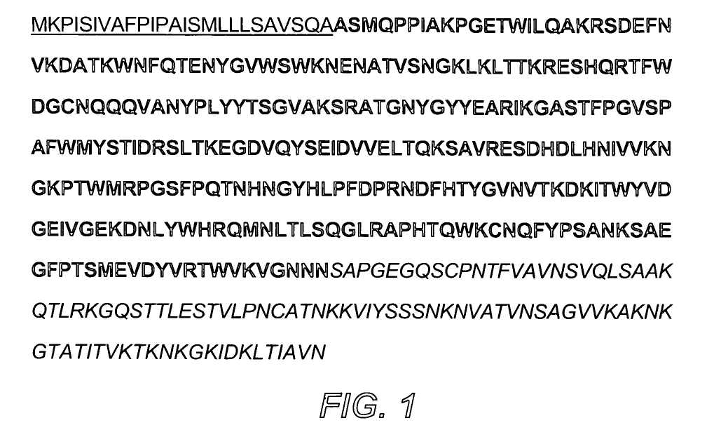

### DESCRIPTION OF THE INVENTION

The present invention provides cleaning compositions comprising at least one carrageenase enzyme, methods for producing carrageenase enzymes in host cells, host cells comprising recombinant polynucleotides encoding at least one carrageenase, and recombinant polynucleotides encoding carrageenase.

### Definitions

Unless defined otherwise herein, all technical and scientific terms used herein have the same meaning as commonly understood by one of ordinary skill in the art to which this invention belongs. Various scientific dictionaries that include the terms included herein are well known and available to those in the art. Although any methods and materials similar or equivalent to those described herein find use in the practice or testing of the present invention, the preferred methods and materials are described. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation and amino acid sequences are written left to right in amino to carboxy orientation, respectively. It is to be understood that this invention is not limited to the particular methodology, protocols, and reagents described, as these may vary, depending upon the context they are used by those of skill in the art.

All patents and publications, including all sequences disclosed within such patents and publications, referred to herein are expressly incorporated by reference, to the same extent as if each individual publication or patent application was specifically and individually indicated to be incorporated by reference. All documents cited are, in relevant part, incorporated herein by reference; the citation of any document is not to be construed as an admission that it is prior art with respect to the present invention.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the Specification as a whole. Accordingly, as indicated above, the terms defined immediately below are more fully defined by reference to the specification as a whole.

The term “promoter” is defined herein as a nucleic acid that directs transcription of a downstream polynucleotide in a cell. In certain cases, the polynucleotide contains a coding sequence and the promoter directs the transcription of the coding sequence into translatable RNA.

The term “coding sequence” is defined herein as a nucleic acid that, when placed under the control of appropriate control sequences including a promoter, is transcribed into mRNA which can be translated into a polypeptide. In some embodiments, coding sequences comprise a single open reading frame, while in other embodiments, coding sequences comprise several open reading frames separated by introns, for example. A coding sequence may be cDNA, genomic DNA, synthetic DNA or recombinant DNA, for example. A coding sequence generally starts at a start codon (e.g., ATG) and ends at a stop codon (e.g., UAA, UAG and UGA).

The term “recombinant” refers to a polynucleotide or polypeptide that does not naturally occur in a host cell. In some embodiments, recombinant molecules comprise two or more naturally occurring sequences that are linked together in a way that does not occur naturally. In some instances, the recombinant polynucleotide comprises a naturally-occurring sequence and a synthetic sequence.

As used herein, the terms “carrageenase” and “carrageenase protein” refer to a serine glycoside hydrolase of the family 16 (GH16) of glycosyl hydrolases. Carrageenase protein has an activity described as EC 3.2.1.83, according to IUMBM enzyme nomenclature. The activity of exemplary carrageenase proteins is generally described in Michel et al., Appl Microbiol Biotechnol 71:23-33 (2006). In some particularly preferred embodiments, the carrageenase of the present invention is a kappa-carrageenase (“k-carrageenase” or “CgkA”).

Unless otherwise indicated, all amino acid positions in a carrageenase protein are relative the amino acid sequence set forth in SEQ ID NO:2. In some embodiments, comparisons of carrageenase proteins are made by alignment of the carrageenase protein with SEQ ID NO:2 using the BLASTP program (See e.g., Altschul, Nucl. Acids Res., 25:3389-3402 [1997); and Schäffer, Bioinformatics 15:1000-1011 [1999]) under default conditions, as available from the world wide website of the National Center of Biotechnology Information (NCBI). In some embodiments, comparisons of carrageenase proteins are made by alignment of the carrageenase protein with SEQ ID NO:4 using the BLASTP program. SEQ ID NO:1 encodes the unprocessed mature form of the carrageenase, which comprises the mature portion and the pro-portion (SEQ ID NO:2); and SEQ ID NO:3 encodes the mature active form of the carrageenase (SEQ ID NO:4). It is the mature active form that is produced by the methods of the invention, and that is included in the cleaning compositions of the invention. A “full-length” carrageenase comprises a secretory signal peptide, a mature portion and a C-terminal pro-portion.

“Naturally-occurring” or “wild-type” refers to a carrageenase protein or a polynucleotide encoding a carrageenase protein having the unmodified amino acid sequence identical to that found in nature. Naturally occurring enzymes include native enzymes, such as those enzymes naturally expressed or found in the particular microorganism. A sequence that is wild-type or naturally-occurring refers to a sequence from which a variant, or a synthetic sequence is derived. The wild-type sequence may encode either a homologous or heterologous protein.

As used herein, “synthetic” refers to molecule that is produced by in vitro chemical or enzymatic synthesis. It includes, but is not limited to, carrageenase variant nucleic acids made with optimal codon usage for host organisms, such as but not limited to Bacillus.

The terms “signal sequence,” “signal peptide” and “secretory signal peptide” refer to any sequence of nucleotides and/or amino acids which may participate in the secretion of the mature or precursor forms of the protein. This definition of signal sequence is a functional one, meant to include all those amino acid sequences encoded by the N-terminal portion of the protein gene, which participate in the effectuation of the secretion of the protein.

The terms “pro sequence” and “pro region” refer to an amino acid sequence at the C-terminus of the mature form of the carrageenase that is necessary for the secretion/production of the carrageenase. Cleavage of the pro sequence results in a mature active carrageenase. To exemplify, a pro region of the carrageenase of the present invention includes the amino acid sequence identical to the N-terminal residues of SEQ ID NO:2 from amino acid 277 to amino acid 372 (i.e., SAPGEGQSCPNTFVAVNSVQLSAAKQTLRKGQSTTLESTVLPNCATNKKVIYSSSN KNVATVNSAGVVKAKNKGTATITVKTKNKGKIDKLTIAVN; SEQ ID NO:5)

The terms “mature form” and “mature region” refer to the final functional portion of the protein. To exemplify, a mature form of the carrageenase of the present invention at least includes the amino acid sequence identical to residues 1-277 of SEQ ID NO: 2 and SEQ ID NO: 4. In this context, the “mature form” is “processed from” a full-length carrageenase, wherein the processing of the full-length carrageenase encompasses the removal of the signal peptide and the removal of the pro region.

As used herein, “expression vector” refers to a DNA construct containing a DNA sequence that is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. In the present specification, “plasmid,” “expression plasmid,” and “vector” are often used interchangeably as the plasmid is the most commonly used form of vector at present. However, the invention is intended to include such other forms of expression vectors that serve equivalent functions and which are, or become, known in the art. “Vectors” include cloning vectors, expression vectors, shuttle vectors, plasmids, phage or virus particles, DNA constructs, cassettes and the like. Expression vectors may include regulatory sequences such as promoters, signal sequences, a coding sequences and transcription terminators.

The term “operably linked” refers to an arrangement of elements that allows them to be functionally related. For example, a promoter is operably linked to a coding sequence if it controls the transcription of the sequence, and a signal sequence is operably linked to a protein if the signal sequence directs the protein through the secretion system of a host cell.

The term “promoter/enhancer” denotes a segment of DNA which contains sequences capable of providing both promoter and enhancer functions (for example, the long terminal repeats of retroviruses contain both promoter and enhancer functions). The enhancer/promoter may be “endogenous” or “exogenous” (or “heterologous”). An endogenous enhancer/promoter is one which is naturally linked with a given gene in the genome. An exogenous (heterologous) enhancer/promoter is one which is placed in juxtaposition to a gene by means of genetic manipulation (i.e., molecular biological techniques).

The term “nucleic acid” encompasses DNA, RNA, single or doubled stranded and modifications thereof. The terms “nucleic acid” and “polynucleotide” may be used interchangeability herein.

The term “DNA construct” as used herein means a nucleic acid sequence that comprises at least two DNA polynucleotide fragments.

The term “production” with reference to a carrageenase, encompasses the two processing steps of a full-length carrageenase, including: the removal of the signal peptide, which is known to occur during protein secretion; and the removal of the pro region, which creates the active mature form of the enzyme and which is known to occur during the maturation process (Wang et al., Biochemistry 37:3165-3171 [1998); Power et al., Proc Natl Acad Sci USA 83:3096-3100 [1986)).

As used herein, the terms “polypeptide” and “protein” are used interchangeably and include reference to a polymer of any number of amino acid residues. The terms apply to amino acid polymers in which one or more amino acid residue is an artificial chemical analog of a corresponding naturally occurring amino acid, as well as to naturally occurring amino acid polymers. The terms also apply to polymers containing conservative amino acid substitutions such that the polypeptide remains functional. “Peptides” are polypeptides having less than 50 amino acid residues.

A “host cell” is a cell which that contains a recombinant nucleic acid in its genome (i.e., the recombinant nucleic acid is an integrant) and/or in an extrachromosomal vector that replicates autonomously from the genome of the host cell. Any suitable cell type finds use as a host cell in the present invention. As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or prepro-form of the protein variant, such variants, when expressed, are typically secreted from the host cell into the culture medium.

“Transformation” means introducing DNA into a cell so that the DNA is maintained in the cell either as an extrachromosomal element or chromosomal integrant. The term “introduced” in the context of inserting a nucleic acid sequence into a cell, including methods including transformation, transduction or transfection. Means of transformation include, but are not limited to protoplast transformation, calcium chloride precipitation, electroporation, naked DNA and the like as known in the art. (See, Chang and Cohen, Mol. Gen. Genet., 168:111-115 [1979]; Smith et al., Appl. Env. Microbiol., 51:634 [1986]; and the review article by Ferrari et al., in Harwood, Bacillus, Plenum Publishing Corporation, pp. 57-72 [1989]).

An “inactivated gene” is a locus of a genome that, prior to its inactivation, was capable of producing a protein (i.e., capable of being transcribed into an RNA that can be translated to produce a full length, catalytically active, polypeptide). A gene is inactivated when it not transcribed and translated into a full length catalytically active protein. A gene may be inactivated by altering a sequence required for its transcription, by altering a sequence required for RNA processing, (e.g., poly-A tail addition), and/or by altering a sequence required for translation. A deleted gene, a gene containing a deleted region, a gene containing a rearranged region, a gene having an inactivating point mutation or frameshift and a gene containing an insertion are types of inactivated genes. A gene may also be inactivated using antisense, RNA interference or any other method that abolishes expression of that gene.

The terms “recovered,” “isolated,” and “separated,” as used herein refer to a protein, cell, nucleic acid or amino acid that is removed from at least one component with which it is naturally associated.

As used herein, “culturing” refers to growing a population of microbial cells under suitable conditions in a liquid, solid or semi-solid medium. In some embodiments, “culturing” refers to fermentative recombinant production of an exogenous protein of interest and/or other desired end products. Typically, fermentation occurs in a vessel or reactor.

The term “carrageenans” herein refers to 1,3-α-1,4-β-galactans from the cell walls of red algae (Rhodophycae), substituted by one (κ-), two (ι-) or three (λ-carrageenan) sulfated groups per disaccharide monomer.

As used herein, unless otherwise indicated, “cleaning compositions” and “cleaning formulations,” refer to compositions that find use in the removal of undesired compounds from items to be cleaned, such as fabric, dishes, contact lenses, other solid substrates, hair (shampoos), skin (soaps and creams), teeth (mouthwashes, toothpastes), etc. The term encompasses any materials/compounds selected for the particular type of cleaning composition desired and the form of the product (e.g., liquid, gel, granule, or spray compositions), as long as the composition is compatible with the carrageenase and other enzyme(s) used in the composition. The specific selection of cleaning composition materials is readily made by considering the surface, item or fabric to be cleaned, and the desired form of the composition for the cleaning conditions during use. These terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. Indeed, it is intended that in addition to carrageenase, the term encompasses detergents that contain surfactants, transferase(s), hydrolytic enzymes, oxido reductases, builders, bleaching agents, bleach activators, bluing agents and fluorescent dyes, caking inhibitors, masking agents, enzyme activators, antioxidants, and solubilizers.

As used herein the term “surface cleaning composition,” refers to detergent compositions for cleaning hard surfaces such as floors, walls, tile, bath and kitchen fixtures, and the like. Such compositions are provided in any form, including but not limited to solids, liquids, emulsions, etc.

As used herein, “dishwashing composition” refers to all forms for compositions for cleaning dishes, including but not limited to granular and liquid forms.

As used herein, “fabric cleaning composition” refers to all forms of detergent compositions for cleaning fabrics, including but not limited to, granular, liquid and bar forms.

As used herein, “fabric” encompasses any textile material. Thus, it is intended that the term encompass garments, as well as fabrics, yarns, fibers, non-woven materials, natural materials, synthetic materials, and any other textile material.

As used herein, “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, “textile materials” is a general term for fibers, yarn intermediates, yarn, fabrics, and products made from fabrics (e.g., garments and other articles).

As used herein, “effective amount of enzyme” refers to the quantity of enzyme necessary to achieve the enzymatic activity required in the specific application (e.g., personal care product, cleaning composition, etc.). Such effective amounts are readily ascertained by one of ordinary skill in the art and are based on many factors, such as the particular enzyme variant used, the cleaning application, the specific composition of the cleaning composition, and whether a liquid or dry (e.g., granular, bar) composition is required, and the like.

As used herein, the terms “purified” and “isolated” refer to the removal of contaminants from a sample. For example, carrageenases are purified by removal of contaminating proteins and other compounds within a solution or preparation that are not carrageenase. In some embodiments, recombinant carrageenase are expressed in bacterial or fungal host cells and these recombinant carrageenases are purified by the removal of other host cell constituents; the percent of recombinant carrageenase polypeptides is thereby increased in the sample. In some particularly preferred embodiments, the carrageenase of the present invention is substantially purified to a level of at least about 99% of the protein component, as determined by SDS-PAGE or other standard methods known in the art. In some alternative preferred embodiments, the carrageenase of the present invention comprises at least about 99% of the carrageenase component of the compositions. In yet some alternative embodiments, the carrageenase is present in a range of about at least 90-95% of the total protein and/or carrageenase component.

As used herein, functionally and/or structurally similar proteins are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms (e.g., a bacterial enzyme and a fungal enzyme). In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the carrageenase of the present invention). In addition, the term “related proteins” encompasses tertiary structural homologs and primary sequence homologs (e.g., the carrageenase of the present invention). For example, the present invention encompasses such homologues including but not limited to such enzymes as the carrageenases of Zobellia galactanivorans, Bacillus circulans and Strongylocentrotus pupuratus, and Cytophaga drobachiensis.

Related (and derivative) proteins comprise “variant proteins.” In some preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In some preferred embodiments, the number of different amino acids between variants is between 1 and 10. In some particularly preferred embodiments, related proteins and particularly variant proteins comprise at least about 70%, about 75%, about 80%, about 85%, about 90%, about 95%, about 97%, about 98%, or about 99% amino acid sequence identity.

In some preferred embodiments of the present invention, the carrageenase gene is ligated into an appropriate expression plasmid. The cloned carrageenase gene is then used to transform or transfect a host cell under conditions such that the carrageenase gene is expressed. This plasmid may replicate in hosts in the sense that it contains the well-known elements necessary for plasmid replication or the plasmid may be designed to integrate into the host chromosome. The necessary elements are provided for efficient gene expression (e.g., a promoter operably linked to the gene of interest). In some embodiments, these necessary elements are supplied as the gene's own homologous promoter if it is recognized, (i.e., transcribed by the host), a transcription terminator (e.g., a polyadenylation region for eukaryotic host cells) which is exogenous or is supplied by the endogenous terminator region of the carrageenase gene. In some embodiments, a selection gene such as an antimicrobial resistance gene that enables continuous cultural maintenance of plasmid-infected host cells by growth in antimicrobial-containing media is also included.

The terms “nucleic acid molecule encoding,” “nucleic acid sequence encoding,” “DNA sequence encoding,” “DNA encoding” and “polynucleotide encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, “homologous protein” refers to a protein (e.g., carrageenase) that has similar action and/or structure, as a kappa-carrageenase (e.g., carrageenase from another source). It is not intended that homologs be necessarily related evolutionarily. Thus, it is intended that the term encompass the same or similar enzyme(s) (i.e., in terms of structure and function) obtained from different species. In some embodiments, it is desirable to identify a homolog that has a quaternary, tertiary and/or primary structure similar to the protein of interest.

As used herein, “homologous genes” refers to at least a pair of genes from different species, which genes correspond to each other and which are identical or very similar to each other. The term encompasses genes that are separated by speciation (i.e., the development of new species) (e.g., orthologous genes), as well as genes that have been separated by genetic duplication (e.g., paralogous genes). These genes encode “homologous proteins.”

As used herein, “ortholog” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

For example, PILEUP is a useful program to determine sequence homology levels. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps. Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). One particularly useful BLAST program is the WU-BLAST-2 program (See, Altschul et al., Meth. Enzymol., 266:460-480 [1996]). parameters “W,” “T,” and “X” determine the sensitivity and speed of the alignment. The BLAST program uses as defaults a wordlength (W) of 11, the BLOSUM62 scoring matrix (See, Henikoff and Henikoff, Proc. Natl. Acad. Sci. USA 89:10915 [1989]) alignments (B) of 50, expectation (E) of 10, M′5, N′-4, and a comparison of both strands.

As used herein, “percent (%) nucleic acid sequence identity” is defined as the percentage of nucleotide residues in a candidate sequence that is identical with the nucleotide residues of the sequence. Similarly, “percent amino acid sequence identity” herein refers to the percentage of amino acid residues in a candidate sequence that is identical with the nucleotide residues of the sequence.

### Carrageenans and Carrageenases

Carrageenans represent one of the major texturizing ingredients in the food industry. Carrageenan is a generic name for a family of linear, sulfated galactans, obtained by extraction from certain species of marine red algae (Rhodophyta). They are composed of D-galactose residues linked by alternating alternating a (1→3) and β (1→4) linkages. Depending on the presence of a 3,6-anhydro bridge in the α (1→4)-linked galactose residue and on the position of sulfate substituents, they are referred to as κ-, ι-, and λ-carrageenans (Kloareg and Quatrano, Oceanogr. Mar. Biol. Annu. Rev., 26, 259-315 [1988)). In addition to these three major carrageenan types, two other types, called μ- and v-carrageenan, are often encountered in commercial carrageenan samples and are the biological precursors of, respectively, κ- and ι-carrageenan (van de Velde et al., Carbohydrate Res. 339:2309-2313 [2004)).

The three major forms of carrageenans are often listed among ingredients on diverse processed food, cosmetics, and pharmaceutical products. The kappa-, iota- and lambda-carrageenans are used either singly, in combination with each other, or with other types of dietary fiber. Carrageenans are used in dairy food products such as ice cream, yogurt, flavored milks, whipped toppings, puddings, cheeses and sour cream. In these products, carrageenans may be used for meltdown control, bodying, fat and protein stabilization, suspension and emulsion stabilization, gelation, thickening, prevention of whey separation, and syneresis control. In processed poultry, ham, and red meat products, carrageenans increase the yield by trapping water in the meat. Carrageenans have also been used to develop a carrageenan-based fat-replacer. Other food industry applications include texture modification function in juices, ready to spread icings, jams, jellies, salad dressing, candies, and as browning inhibitors for fresh fruit processing control. Most food products contain kappa-carrageenan, either alone or in combination with another type of carrageenan (See e.g., Shah, and Huffman, Ecol. Food Nutrition 42:357-371 [2003)).

Microorganisms which produce enzymes capable of hydrolyzing iota- and kappa-carrageenans have been isolated (See e.g., Bellion et al., Can. J. Microbiol., 28: 874-80 [1982]; and Yaphe and Baxter, Appl. Microbiol. 3:380-383 [1955]). The Pseudoalteromonas carrageenovora strain (also known as Alteromonas carrageenovora) was shown to possess kappa- and lambda-carrageenase activities. Another group of bacteria capable of degrading carrageenans has been characterized (See e.g., Sarwar et al., J. Gen. Appl. Microbiol., 29:145-55 [1983]). These yellow-orange bacteria are assigned to the Cytophaga group of bacteria and some of these bacteria have the property of hydrolyzing both agar and carrageenans.

The genes encoding kappa-carrageenases that have been cloned include the kappa carrageenase gene cgkA from P. carrageenovora (Barbeyron et al., Gene 139:105-109 [1994]) and the cgkA gene of Cytophaga drobachiensis (Barbeyron et al., Mol. Biol. Evol. 15:528-537 [1998]).

Purification and characterization of several kappa-carrageenases, such as the kappa-carrageenase of Cytophaga drobachiensis (Barbeyron et al., Mol. Biol. Evol., supra), the kappa-carrageenase of Alteromonas carrageenovora, the kappa-carrageenase from Zobelia galactcinovorans, Bacillus circulans and Strongylocentrotus pupuratus have been described (See e.g., Strohmeier et al., Protein Sci., 13:3200-3213 [2004]), and there is a detailed study of the .kappa.-carrageenase of Alteromonas carrageenovora available (See, Potin et al., Eur. J. Biochem., 228, 971-975 [1995]].

The present invention provides an isolated recombinant polynucleotide that comprises a sequence encoding kappa-carrageenase that is operably linked to a heterologous sequence encoding a secretory signal peptide. In some preferred embodiments, the encoded kappa-carrageenase belongs to the family 16 (GH16) of glycosyl hydrolases. In some embodiments, the sequence encoding the mature form of the kappa-carrageenase is a wild-type sequence, such as a sequence derived from species of marine bacteria (e.g., Alteromonas species). In some preferred embodiments, the sequence is the wild-type sequence of the carrrageenase of Alteromonas carrageenovora. Other carrageenase sequences that find use in the present invention include, but are not limited to those encoding the carrageenase from Zobellia galactanivorans, Bacillus circulans. Strongylocentrotus pupuratus and Cytophaga drobachiensis. The invention also encompasses polynucleotide sequences, whether wild-type of synthetic, that are derived from genes homologous to those of A. carrageenovora, Z. galactanivorans, B. circulans and S. pupuratus and C. drobachiensis. Thus, the invention encompasses carrageenase enzymes encoded by genes that are homologous to that of the kappa-carrageenase gene of A. carrageenovora. As indicated above, “homologous genes” are genes that correspond to each other and which are identical or very similar to each other, yet are obtained from different species. The term encompasses genes that are separated by speciation (i.e., the development of new species) (e.g., orthologous genes), as well as genes that have been separated by genetic duplication (e.g., paralogous genes). Homologous genes encode for homologous proteins.

The invention also encompasses polynucleotides encoding carrageenase proteins that are related by being structurally and/or functionally similar. In some embodiments, these proteins are derived from a different genus and/or species, including differences between classes of organisms (e.g., a bacterial protein and a fungal protein). In some embodiments, these proteins are derived from a different genus and/or species. In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the carrageenase of the present invention). For example, the present invention encompasses such homologues including but not limited to such enzymes as the carrageenases of Z. galactanivorans, B. circulans, S. pupuratus and C. drobachiensis.

As indicated above, related (and derivative) proteins comprise “variant proteins.” In some preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In some preferred embodiments, the number of different amino acids between variants is between 1 and 10. In some particularly preferred embodiments, related proteins and particularly variant proteins comprise at least about 70%, about 75%, about 80%, about 85%, about 90%, about 95%, about 97%, about 98%, or about 99% amino acid sequence identity. Certain carrageenase enzymes of interest have an activity described as EC3.2.1.83 according to IUMBM enzyme nomenclature. Several methods are known in the art that are suitable for generating variants of the enzymes of the present invention, including but not limited to site-saturation mutagenesis, scanning mutagenesis, insertional mutagenesis, random mutagenesis, site-directed mutagenesis, and directed-evolution, as well as various other recombinatorial approaches.

Characterization of wild-type and mutant proteins is accomplished via any means suitable and is preferably based on the assessment of properties of interest. For example, pH and/or temperature, as well as detergent and/or oxidative stability is/are determined in some embodiments of the present invention. Indeed, it is contemplated that enzymes having various degrees of stability in one or more of these characteristics (pH, temperature, proteolytic detergent stability, and/or oxidative stability) will find use.

In certain preferred embodiments, the recombinant polunucleotides of the invention comprise a polynucleotide sequence that may be codon optimized for expression of a carrageenase fusion protein in the host cell used. Since codon usage tables listing the usage of each codon in many cells are known in the art (e.g., Nakamura et al., Nucl. Acids Res., 28:292 [2000]) or readily derivable, such nucleic acids can be readily designed giving the amino acid sequence of a protein to be expressed. In some embodiments, the codon-optimized sequence comprises a polynucleotide that encodes the mature form of the carrageenase and the N-terminal pro-region. In some embodiments, the codon-optimized sequence is at least about 70% identical to SEQ ID NO:1, shown below.

(SEQ ID NO: 1)

GCTAGCATGCAACCACCTATCGCTAAACCAGGAGAAACATGGATTCTTCA
AGCAAAACGTTCTGATGAATTTAACGTTAAAGACGCTACTAAATGGAACT
TCCAAACAGAAAACTATGGTGTATGGTCTTGGAAAAACGAAAATGCAACT
GTTTCAAACGGTAAACTTAAATTAACTACAAAACGTGAATCTCACCAAAG
AACATTCTGGGATGGTTGCAACCAACAACAAGTTGCAAACTACCCACTTT
ATTACACTTCTGGTGTTGCAAAATCACGTGCTACAGGAAACTACGGTTAT
TACGAAGCACGTATCAAAGGAGCATCTACTTTCCCTGGTGTATCTCCAGC
TTTCTGGATGTACTCTACAATTGACCGTAGCCTTACTAAAGAAGGTGACG
TTCAATACTCTGAAATCGACGTAGTTGAACTTACACAAAAATCAGCAGTT
CGTGAATCTGACCACGATCTTCACAACATTGTAGTTAAAAACGGTAAACC
TACATGGATGCGCCCGGGTTCTTTTCCTCAAACTAACCATAACGGCTACC
ACCTTCCATTTGATCCTCGTAACGACTTCCACACATACGGAGTTAACGTA
ACTAAAGATAAAATCACATGGTATGTTGACGGTGAAATTGTAGGAGAAAA
AGACAACCTTTATTGGCACCGTCAAATGAACTTAACTCTTTCTCAAGGCC
TTAGAGCGCCTCACACACAATGGAAATGCAACCAATTCTACCCATCAGCA
AACAAATCTGCTGAAGGTTTCCCTACTTCAATGGAAGTAGACTACGTTCG
TACATGGGTTAAAGTAGGAAACAACAATTCTGCACCAGGTGAAGGACAAT
CATGTCCTAACACATTCGTTGCTGTAAACTCTGTTCAACTTTCAGCTGCA
AAACAAACTCTTCGTAAAGGTCAATCTACAACTTTAGAATCAACTGTTCT
TCCAAACTGCGCAACAAACAAAAAAGTTATCTACTCTAGCTCAAACAAAA
ACGTAGCTACTGTTAACTCTGCAGGTGTTGTAAAAGCAAAAAACAAAGGT
ACAGCTACTATTACAGTTAAAACAAAAAACAAAGGAAAAATCGATAAACT
TACAATCGCAGTAAAC

In other embodiments, the codon-optimized sequence is at least about 75% identical to SEQ ID NO:1, or more preferably about 80%, about 85%, about 90%, about 95%, about 97%, about 98%, or about 99% identical to SEQ ID NO:1.

In some other embodiments, the codon-optimized sequence comprises a polynucleotide that encodes the mature form of the carrageenase. In some embodiments, the codon-optimized sequence is at least about 70% identical to SEQ ID NO: 3, as provided below:

(SEQ ID NO: 3)

GCTAGCATGCAACCACCTATCGCTAAACCAGGAGAAACATGGATTCTTCA
AGCAAAACGTTCTGATGAATTTAACGTTAAAGACGCTACTAAATGGAACT
TCCAAACAGAAAACTATGGTGTATGGTCTTGGAAAAACGAAAATGCAACT
GTTTCAAACGGTAAACTTAAATTAACTACAAAACGTGAATCTCACCAAAG
AACATTCTGGGATGGTTGCAACCAACAACAAGTTGCAAACTACCCACTTT
ATTACACTTCTGGTGTTGCAAAATCACGTGCTACAGGAAACTACGGTTAT
TACGAAGCACGTATCAAAGGAGCATCTACTTTCCCTGGTGTATCTCCAGC
TTTCTGGATGTACTCTACAATTGACCGTAGCCTTACTAAAGAAGGTGACG
TTCAATACTCTGAAATCGACGTAGTTGAACTTACACAAAAATCAGCAGTT
CGTGAATCTGACCACGATCTTCACAACATTGTAGTTAAAAACGGTAAACC
TACATGGATGCGCCCGGGTTCTTTTCCTCAAACTAACCATAACGGCTACC
ACCTTCCATTTGATCCTCGTAACGACTTCCACACATACGGAGTTAACGTA
ACTAAAGATAAAATCACATGGTATGTTGACGGTGAAATTGTAGGAGAAAA
AGACAACCTTTATTGGCACCGTCAAATGAACTTAACTCTTTCTCAAGGCC
TTAGAGCGCCTCACACACAATGGAAATGCAACCAATTCTACCCATCAGCA
AACAAATCTGCTGAAGGTTTCCCTACTTCAATGGAAGTAGACTACGTTCG
TACATGGGTTAAAGTAGGAAACAACAAT

In some other embodiments, the codon-optimized sequence is at least about 75% identical to SEQ ID NO:3, or more preferably about 80%, about 85%, about 90%, about 95%, about 97%, about 98%, or about 99% identical to SEQ ID NO:3.

In some preferred embodiments, the codon-optimized sequence is operably linked to a polynucleotide sequence that encodes a secretory signal peptide to encode a carrageenase fusion protein. The choice of signal sequence, promoter and terminator largely depends upon the host cell used. As noted above, in certain embodiments, a Bacillus host cell is employed, in which the signal sequence may be any sequence of amino acids that is capable of directing the fusion protein into the secretory pathway of the Bacillus host cell. In certain cases, signal sequences that may be employed include the signal sequences of proteins that are secreted from wild-type Bacillus cells. Such signal sequences include the signal sequences encoded by α-amylase, protease, (e.g., aprE or subtilisin E), or β-lactamase genes. In some preferred embodiments, the recombinant polynucleotides of the invention comprise a polynucleotide encoding an AprE signal peptide. Other exemplary signal sequences include, but are not limited to, the signal sequences encoded by an α-amylase gene, a subtilisin gene, a β-lactamase gene, a neutral protease gene (e.g., nprT, nprS, nprM), or a prsA gene from any suitable Bacillus species, including, but not limited to B. stearothermophilus, B. licheniformis, B. lentus, B. subtilis, and B. amyloliquefaciens. In some embodiments, the signal sequence is encoded by the aprE gene of B. subtilis (See e.g., Appl. Microbiol. Biotechnol., 62:369-73 [2003]). Further signal peptides find use in the present invention (See e.g., Simonen and Palva, Micro. Rev., 57:109-137 [1993]; etc.).

In certain embodiments, the recombinant polynucleotide of the invention is contained in an expression cassette for expressing a carrageenase in a host cell. As such, in some particular embodiments, the expression cassette comprises, in operable linkage: a promoter, a coding sequence encoding a carrageenase protein (which carrageenase protein may be contained in a fusion protein comprising a signal sequence and said carrageenase protein), and a terminator sequence.

In some preferred embodiments, the expression cassette comprising a wild-type or a synthetic carrageenase gene is ligated into an appropriate expression plasmid. The carrageenase gene is then used to transform or transfect a host cell in order to express the carrageenase gene. In some embodiments, this plasmid replicates in host cells in the sense that it contains the well-known elements necessary for plasmid replication, while in other embodiments, the plasmid is designed to integrate into the host chromosome. The necessary elements are provided for efficient gene expression (e.g., a promoter operably linked to the gene of interest). In some embodiments, these necessary elements are supplied as the gene's own homologous promoter if it is recognized, (i.e., transcribed, by the host), a transcription terminator (a polyadenylation region for eukaryotic host cells) which is exogenous or is supplied by the endogenous terminator region of the carrageenase gene. In some embodiments, a selection gene such as an antibiotic resistance gene that enables continuous cultural maintenance of plasmid-infected host cells by growth in antimicrobial-containing media is also included.

The choice of signal sequence, promoter and terminator largely depend on the host cell used. As noted above, in certain embodiments, a Bacillus host cell is employed, the signal sequence may be any sequence of amino acids that is capable of directing the fusion protein into the secretory pathway of the Bacillus host cell. In certain cases, signal sequences that may be employed include the signal sequences of proteins that are secreted from wild-type Bacillus cells. Such signal sequences include the signal sequences encoded by α-amylase, protease, (e.g., aprE or subtilisin E), or β-lactamase genes. Exemplary signal sequences include, but are not limited to, the signal sequences encoded by an α-amylase gene, an subtilisin gene, a β-lactamase gene, a neutral protease gene (e.g., nprT, nprS, nprM), or a prsA gene from any suitable Bacillus species, including, but not limited to B. stearothermophilus, B. licheniformis, B. lentus, B. subtilis, and B. amyloliquefaciens. In some embodiments, the signal sequence is encoded by the aprE gene of B. subtilis (See e.g., Appl. Microbiol. Biotechnol., 62:369-73 [2003]). Further signal peptides find use in the present invention (See e.g., Simonen and Palva, Micro. Rev., 57:109-137 [1993]; etc.).

Suitable promoters and terminators for use in Bacillus cells are known and include, but are not limited to: the promoters and terminators of apr (alkaline protease), npr (neutral protease), amy (α-amylase) and β-lactamase genes, as well as the B. subtilis levansucrase gene (sacB), B. licheniformis alpha-amylase gene (amyL), B. stearothermophilus maltogenic amylase gene (amyM), B. amyloliquefaciens alpha-amylase gene (amyQ), B. licheniformis penicillinase gene (penP), B. subtilis xylA and xylB genes, the promoters and terminators described in WO 93/10249, WO 98/07846, and WO 99/43835. In some preferred embodiments, the expression cassette for expressing the carrageenase protein comprises the aprE promoter and the LAT terminator sequences (Yuuki et al., J. Bioche. 98:1147-1156 [1985]).

In some particular embodiments, the recombinant nucleic acid further contains a selectable marker for the selection of cells that contain the recombinant nucleic acid over other cells that do not contain the recombinant nucleic acid. Exemplary selectable markers are described in the references cited in the previous paragraph, and include, but are not limited to, selectable markers that provide resistance to antimicrobials, (e.g., resistance to hygromycin, bleomycin, chloroamphenicol, phleomycin, kanamycin, streptomycin, ampicillin, tetracycline, thiostrepton, etc.).

In some certain preferred embodiments, the coding sequence is codon optimized for expression of the fusion protein in the host cell used. Since codon usage tables listing the usage of each codon in many cells are known in the art (See e.g., Nakamura et al., Nucl. Acids Res., 28: 292 [2000]) or readily derivable, such nucleic acids can be readily designed giving the amino acid sequence of a protein to be expressed.

The recombinant polynucleotide of the invention may be present, (e.g., integrated), into a genome (e.g., the nuclear genome) of a host cell, or may be present in a vector, (e.g., a phage, plasmid, viral, or retroviral vector), that autonomously replicates in the host cell. In some certain embodiments, the vector is an expression vector for expressing a protein in a host cell. Vectors and systems for expression of recombinant proteins in Bacillus host cells are well known in the art.

### Host Cells

The present invention also provides a host cell comprising a recombinant polynucleotide encoding at least one carrageenase. As discussed above, in some embodiments, the recombinant polynucleotide of the invention is contained in an expression cassette comprising a suitable promoter, a signal sequence, a polynucleotide encoding a carrageenase and a terminator sequence. In some embodiments, the polynucleotide sequence encoding the carrageenase enzyme is the wild-type sequence of the carrrageenase of A. carrageenovora. The invention also provides an expression cassette that comprises wild-type carrageenase sequences that include but are not limited to those encoding the carrageenase from Z. galactanivorans, B. circulans, S. pupuratus. and C. drobachiensis. In some preferred embodiments, the invention provides an expression cassette that comprises a codon-optimized sequence encoding a kappa-carrageenase (e.g., SEQ ID NO:1 or SEQ ID NO:3). In some embodiments, the codon-optimized sequence comprises a polynucleotide that encodes the mature form of the carrageenase and the N-terminal pro-region. In some preferred embodiments, the codon-optimized sequence is at least about 70% identical to SEQ ID NO:1. In some other embodiments, the codon-optimized sequence is at least about 75% identical to SEQ ID NO:1, or more preferably about 80%, about 85%, about 90%, about 95%, about 97%, about 98%, or about 99% identical to SEQ ID NO:1. In some other embodiments, the codon-optimized sequence comprises a polynucleotide that encodes the mature form of the carrageenase. In some additional embodiments, the codon-optimized sequence is at least about 70% identical to SEQ ID NO:3. In still further embodiments, the codon-optimized sequence is at least about 75% identical to SEQ ID NO:3, or more preferably about 80%, about 85%, about 90%, about 95%, about 97%, about 98%, or about 99% identical to SEQ ID NO: 3.

In some embodiments, the invention provides Gram-positive host cells capable of expressing the recombinant polynucleotide of the invention. In some embodiments, the Gram positive host cell comprises a mutation and/or deletion of part or all of the gene encoding a protease, which results in the inactivation of the protease proteolytic activity, either alone or in combination with mutations in other proteases, such as apr, npr, epr, mpr, bpf or isp, or other proteases known to those of skill in the art. In some embodiments of the present invention, the Gram positive microorganism is a member of the genus Bacillus. Any suitable member of the genus Bacillus finds use in the present invention, including, but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. In some preferred embodiments, the Bacillus is B. subtilis. In some embodiments, the host cell comprises a strain that has a history of use for production of proteins with GRAS (i.e., “Generally Recognized as Safe” by the FDA) status.

As noted above, in some particularly preferred embodiments, the Bacillus host cell comprises two or more inactivated protease genes. In some embodiments, the Bacillus host cell contains two inactivated protease genes (See e.g., U.S. Pat. No. 5,387,521) while in other embodiments, the Bacillus host cell contains 5 inactivated protease genes: nprE, aprE, epr, ispA, and bpr genes (See e.g., US20050202535). Since the sequence of the entire B. subtilis genome is publicly available and annotated (See e.g., Moszer, FEBS Lett., 430:28-36 [1998]), the proteases of B. subtilis have been identified and reviewed in detail (See e.g., He et al., Res. Microbiol., 142:797-803 [1991]). In addition, gene disruption methods for Bacillus cells are generally well known in the art (See e.g., Lee et al., Appl. Environ. Microbiol., 66: 476-480 [2000]; Ye et al., Proc. Internatl. Symp. Rec. Adv. Bioindustry, Seoul, Korea: The Korean Society for Applied Microbiology, pp. 160-169 [1996]; Wu et al., J. Bacteriol., 173:4952-4958 [1991]; and Sloma et al., J. Bacteriol., 173:6889-6895 [1991]). Thus, the construction of such strains is well within the ability of one of skill in the art.

In some embodiments, the carrageenase protein that is expressed by the host cells of the invention comprises a kappa-carrageenase amino acid sequence having at least about 70% identity to the kappa-carrageenase of SEQ ID NO:2, set forth below:

(SEQ ID NO: 2)

ASMQPPIAKPGETWILQAKRSDEFNVKDATKWNFQTENYGVWSWKNENAT
VSNGKLKLTTKRESHQRTFWDGCNQQQVANYPLYYTSGVAKSRATGNYGY
YEARIKGASTFPGVSPAFWMYSTIDRSLTKEGDVQYSEIDVVELTQKSAV
RESDHDLHNIVVKNGKPTWMRPGSFPQTNHNGYHLPFDPRNDFHTYGVNV
TKDKITWYVDGEIVGEKDNLYWHRQMNLTLSQGLRAPHTQWKCNQFYPSA
NKSAEGFPTSMEVDYVRTWVKVGNNNSAPGEGQSCPNTFVAVNSVQLSAA
KQTLRKGQSTTLESTVLPNCATNKKVIYSSSNKNVATVNSAGVVKAKNKG
TATITVKTKNKGKIDKLTIAVN

In some other embodiments, the carrageenase protein that is expressed by the host cells of the invention is at least about 75% identical to SEQ ID NO:2, or more preferably about 80%, about 85%, about 90%, about 95%, about 97%, about 98%, or about 99% identical to SEQ ID NO:2.

In preferred embodiments, the carrageenase protein that is expressed and produced by the host cell of the invention comprises a kappa-carrageenase amino acid sequence having at least 70% identity to the kappa-carrageenase of SEQ ID NO:4, as set forth below:

(SEQ ID NO: 4)

ASMQPPIAKPGETWILQAKRSDEFNVKDATKWNFQTENYGVWSWKNENAT
VSNGKLKLTTKRESHQRTFWDGCNQQQVANYPLYYTSGVAKSRATGNYGY
YEARIKGASTFPGVSPAFWMYSTIDRSLTKEGDVQYSEIDVVELTQKSAV
RESDHDLHNIVVKNGKPTWMRPGSFPQTNHNGYHLPFDPRNDFHTYGVNV
TKDKITWYVDGEIVGEKDNLYWHRQMNLTLSQGLRAPHTQWKCNQFYPSA
NKSAEGFPTSMEVDYVRTWVKVGNNN

In other preferred embodiments, the carrageenase protein that is expressed by the host cells of the invention is at least about 75% identical to SEQ ID NO:4, more preferably about 80%, about 85%, about 90%, about 95%, about 97% about 98%, or about 99% identical to SEQ ID NO:4.

The invention also encompasses carrageenase proteins that are related by being structurally and/or functionally similar. In some embodiments, these proteins are derived from a different genus and/or species, including differences between classes of organisms (e.g., a bacterial protein and a fungal protein). In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the carrageenase of the present invention). For example, the present invention encompasses such homologues including but not limited to such enzymes as the carrageenases of Z. galactanivorans, B. circulans, S. pupuratus, and C. drobachiensis. Indeed, as indicated above the present invention encompasses variant proteins (e.g., related and derivative proteins), in particular, those carrageenase enzymes having the activity described as EC 3.2.1.83 according to IUMBM enzyme nomenclature. In addition, the present invention provides cell cultures. In some particularly preferred embodiments, the cell cultures comprise a plurality of Bacillus host cells, as described above, and culture medium.

### Protein Production Methods

The present invention also provides methods of using the above-described cells. In some embodiments, the methods include culturing a host cell of the invention to produce a carrageenase protein. In some additional embodiments and as discussed above, the protein is secreted into the culture medium. In yet further embodiments, the methods comprise the step of recovering the protein from the culture medium. Preferred embodiments provide for the production (i.e., secretion) of the mature form of the kappa-carrageenase protein set forth in SEQ ID NO:4. Variants of the carrageenase of SEQ ID NO:4 and related carrageenase proteins as recited herein are also encompassed by the invention.

In some preferred embodiments, the carrageenase protein is recovered from growth media using any convenient method (e.g., by precipitation, centrifugation, affinity, filtration) or any other suitable method known in the art. For example, affinity chromatography (Tilbeurgh et al., FEBS Lett., 16:215 [1984]); ion-exchange chromatographic methods (Goyal et al., Biores. Technol., 36:37 [1991]; Fliess et al., Eur. J. Appl. Microbiol. Biotechnol. 17:314 [1983]; Bhikhabhai et al., J. Appl. Biochem., 6:336 [1984]; and Ellouz et al., Chromatography 396:307 [1987]), including ion-exchange using materials with high resolution power (Medve et al., J. Chromatography A 808:153 [1998]; hydrophobic interaction chromatography (Tomaz and Queiroz, J. Chromatography A 865:123 [1999]; two-phase partitioning (Brumbauer et al., Bioseparation 7:287 [1999]); ethanol precipitation; reverse phase HPLC; chromatography on silica or on a cation-exchange resin such as DEAE; chromatofocusing; SDS-PAGE; ammonium sulfate precipitation; and gel filtration (e.g., using SEPHADEX G-75), find use in the present invention. In some particular embodiments, the detergent-additive protein is used without purification from the other components in the culture medium. In some embodiments, the components of the culture medium are simply concentrated and then used without further purification of the carrageenase protein from the other components of the growth medium in order to produce a cleaning and/or other composition.

In some embodiments, the host cells are cultured under batch, fed-batch or continuous fermentation conditions. Classical batch fermentation methods use a closed system, wherein the culture medium is made prior to the beginning of the fermentation run, the medium is inoculated with the desired organism(s), and fermentation occurs without the subsequent addition of any components to the medium. In certain cases, the pH and oxygen content, but not the carbon source content, of the growth medium are altered during batch methods. The metabolites and cell biomass of the batch system change constantly up to the time the fermentation is stopped. In a batch system, cells usually progress through a static lag phase to a high growth log phase and finally to a stationary phase where growth rate is diminished or halted. If untreated, cells in the stationary phase eventually die. In general terms, the cells in log phase produce most protein.

A variation on the standard batch system is the “fed-batch fermentation” system. In this system, nutrients (e.g., a carbon source, nitrogen source, O₂, or other nutrient) are only added when their concentration in culture falls below a threshold. Fed-batch systems are useful when catabolite repression is apt to inhibit the metabolism of the cells and where it is desirable to have limited amounts of nutrients in the medium. Measurement of the actual nutrient concentration in fed-batch systems is estimated on the basis of the changes of measurable factors such as pH, dissolved oxygen and the partial pressure of waste gases such as CO₂. Batch and fed-batch fermentations are common and known in the art.

Continuous fermentation is an open system where a defined culture medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth and/or end product concentration. For example, in some embodiments, a limiting nutrient such as the carbon source or nitrogen source is maintained at a fixed rate and all other parameters are allowed to moderate. In other systems, a number of factors affecting growth are altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are known to those of skill in the art and find use in the production of the carrageenase of the present invention.

### Methods of Use

The carrageenase protein produced using the above described methods finds use in any product containing a carrageenase, including, but not limited to cleaning compositions, (e.g., fabric cleaning compositions, such as laundry detergents, surface cleaning compositions, dish cleaning compositions and automatic dishwasher detergent compositions; See e.g., WO0001826, which is incorporated by reference herein). In some embodiments, the cleaning composition is a borax-free composition.

In some particular embodiments, the carrageenase protein is used in an carrageenase-containing laundry detergent comprising from about 1% to about 80%, e.g., about 5% to about 50% (by weight) of surfactant, which may be a non-ionic surfactant, cationic surfactant, an anionic surfactant or a zwitterionic surfactant, or any mixture thereof (e.g., a mixture of anionic and nonionic surfactants). Exemplary surfactants include: alkyl benzene sulfonate (ABS), including linear alkyl benzene sulfonate and linear alkyl sodium sulfonate, alkyl phenoxy polyethoxy ethanol (e.g., nonyl phenoxy ethoxylate or nonyl phenol), diethanolamine, triethanolamine and monoethanolamine. Exemplary surfactants that find use in laundry detergents are known in the art (See e.g., U.S. Pat. Nos. 3,664,961, 3,919,678, 4,222,905, and 4,239,659).

The laundry detergent may be in solid, liquid, gel or bar form, and may further contain a buffer such as sodium carbonate, sodium bicarbonate, or detergent builder, bleach, bleach activator, various enzymes, an enzyme stabilizing agent, suds booster, suppresser, anti-tarnish agent, anti-corrosion agent, soil suspending agent, soil release agent, germicide, pH adjusting agent, non-builder alkalinity source, chelating agent, organic or inorganic filler, solvent, hydrotrope, optical brightener, dye or perfumes. In some preferred embodiments, the laundry detergent comprises in addition to the carrageenase of the present invention, at least one further enzyme (e.g., hemicellulase, peroxidase, protease, cellulase, xylanase, lipase, phospholipase, esterase, cutinase, pectinase, keratinase, reductase, oxidase, phenoloxidase, lipoxygenase, ligninase, pullulanase, tannase, pentosanase, mannanase, β-glucanase, arabinosidase, hyaluronidase, chondroitinase, laccase, and amylases, or mixtures thereof).

The carrageenase protein of the present invention finds use in any suitable composition useful for cleaning a variety of surfaces in need of stain removal. Such cleaning compositions include detergent compositions for cleaning hard surfaces, unlimited in form (e.g., liquid, gel, bar and granular formation); detergent compositions for cleaning fabrics, unlimited in form (e.g., granular, liquid, gel and bar formulations); dishwashing compositions (unlimited in form); oral cleaning compositions, unlimited in form (e.g., dentifrice, toothpaste, gel and mouthwash formulations); denture cleaning compositions, unlimited in form (e.g., liquid, gel or tablet); and contact lens cleaning compositions, unlimited in form (e.g., liquid, tablet).

In some embodiments, the cleaning compositions of the present invention comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratinases, reductases, oxidases, phenoloxidases, lipoxygenases, ligninases, pullulanases, tannases, pentosanases, mannanases, β-glucanases, arabinosidases, hyaluronidase, chondroitinase, laccase, and amylases, or mixtures thereof. In some embodiments, a combination of enzymes is used (i.e., a “cocktail”) comprising conventional applicable enzymes like protease, lipase, cutinase and/or cellulase in conjunction with carrageenase is used.

In some embodiments, the cleaning compositions also comprise, in addition to the proteins described herein, one or more cleaning composition materials compatible with the carrageenase protein. As described herein, the term “cleaning composition material,” refers any liquid, solid or gaseous material selected for the particular type of cleaning composition desired and the form of the product (e.g., liquid, granule, bar, spray, stick, paste, gel), which materials are also compatible with the carageenase used in the composition. The specific selection of cleaning composition materials are readily made by considering the surface material to be cleaned, the desired form of the composition for the cleaning condition during use (e.g., through the wash detergent use). As used herein, “non-fabric cleaning compositions” include hard surface cleaning compositions, dishwashing compositions, oral cleaning compositions, denture cleaning compositions and contact lens cleaning compositions.

The carrageenase protein finds use with various conventional ingredients to provide fully-formulated hard-surface cleaners, dishwashing compositions, fabric laundering compositions, and the like. Such compositions find use in the form of liquids, granules, bars and the like. In some embodiments, such compositions are formulated as modern “concentrated” detergents which contain as much as about 30% to about 60% by weight of surfactants.

In some preferred embodiments, the cleaning compositions of the present invention comprise various anionic, nonionic, zwitterionic, etc., surfactants. Such surfactants are typically present at levels of from about 5% to about 35% of the compositions.

A wide variety of other ingredients useful in detergent cleaning compositions also find use in the compositions herein, including other active ingredients, carriers, hydrotropes, processing aids, dyes or pigments, solvents for liquid formulations, etc. If an additional increment of sudsing is desired, suds boosters such as the C₁₀-C₁₆ alkylamides also find use in the compositions, typically at about 1% to about 10% levels.

In some embodiments, detergent compositions comprise water and/or other solvents as carriers. For example, in some embodiments, low molecular weight primary or secondary alcohols (e.g., methanol, ethanol, propanol, and isopropanol) are suitable. Monohydric alcohols are preferred for solubilizing surfactants, but polyols such as those containing from about 2 to about 6 carbon atoms and from about 2 to about 6 hydroxy groups (e.g., 1,3-propanediol, ethylene glycol, glycerine, and 1,2-propanediol) also find use. In such embodiments, the compositions typically contain from about 5% to about 90%, or typically from about 10% to about 50% of such carriers.

In some embodiments, the detergent compositions provided herein are formulated such that during use in aqueous cleaning operations, the wash water will have a pH between about 6.8 and about 11.0. Finished products thus are typically formulated at this range. Techniques for controlling pH at recommended usage levels include the use of buffers, alkalis, acids, etc., and are well known to those skilled in the art.

Various bleaching compounds, such as the percarbonates, perborates and the like, also find use in such compositions, typically at levels from about 1% to about 15% by weight. In some embodiments, such compositions also contain bleach activators such as tetraacetyl ethylenediamine, nonanoyloxybenzene sulfonate, and the like, which are also known in the art. Usage levels typically range from about 1% to about 10% by weight.

Various soil release agents, especially of the anionic oligoester type, various chelating agents, especially the aminophosphonates and ethylenediaminedisuccinates, various clay soil removal agents, especially ethoxylated tetraethylene pentamine, various dispersing agents, especially polyacrylates and polyasparatates, various brighteners, especially anionic brighteners, various suds suppressors, especially silicones and secondary alcohols, various fabric softeners, especially smectite clays, and the like, all find use in various embodiments of the present compositions, at levels ranging from about 1% to about 35% by weight. Standard formularies and published patents contain multiple, detailed descriptions of such conventional materials.

Enzyme stabilizers also find use in the cleaning compositions of the present invention. Such stabilizers include, but are not limited to propylene glycol (preferably from about 1% to about 10%), sodium formate (preferably from about 0.1% to about 1%), and calcium formate (preferably from about 0.1% to about 1%).

The cleaning compositions of the present invention are formulated into any suitable form and prepared by any suitable process chosen by the formulator, (See e.g., U.S. Pat. No. 5,879,584, U.S. Pat. No. 5,691,297, U.S. Pat. No. 5,574,005, U.S. Pat. No. 5,569,645, U.S. Pat. No. 5,565,422, U.S. Pat. No. 5,516,448, U.S. Pat. No. 5,489,392, U.S. Pat. No. 5,486,303, U.S. Pat. No. 4,515,705, U.S. Pat. No. 4,537,706, U.S. Pat. No. 4,515,707, U.S. Pat. No. 4,550,862, U.S. Pat. No. 4,561,998, U.S. Pat. No. 4,597,898, U.S. Pat. No. 4,968,451, U.S. Pat. No. 5,565,145, U.S. Pat. No. 5,929,022, U.S. Pat. No. 6,294,514, and U.S. Pat. No. 6,376,445, all of which are incorporated herein by reference for some non-limiting examples). When formulating the hard surface cleaning compositions and fabric cleaning compositions of the present invention, the formulator may wish to employ various builders at levels from about 5% to about 50% by weight. Typical builders include the 1-10 micron zeolites, polycarboxylates such as citrate and oxydisuccinates, layered silicates, phosphates, and the like. Other conventional builders are listed in standard formularies.

Other optional ingredients include chelating agents, clay soil removal/anti-redeposition agents, polymeric dispersing agents, bleaches, brighteners, suds suppressors, solvents and aesthetic agents.

In some preferred embodiments, the cleaning compositions of the present invention find use in cleaning surfaces and/or fabrics. In some embodiments, at least a portion of the surface and/or fabric is contacted with at least one embodiment of the cleaning compositions of the present invention, in neat form or diluted in a wash liquor, and then the surface and/or fabric is optionally washed and/or rinsed. For purposes of the present invention, “washing” includes, but is not limited to, scrubbing, and mechanical agitation. In some embodiments, the fabric comprises any fabric capable of being laundered in normal consumer use conditions. In some preferred embodiments, the cleaning compositions of the present invention are used at concentrations of from about 500 ppm to about 15,000 ppm in solution. In some embodiments in which the wash solvent is water, the water temperature typically ranges from about 5° C. to about 70° C. In some preferred embodiments for fabric cleaning, the water to fabric mass ratio is typically from about 1:1 to about 30:1.

In order to further illustrate the present invention and advantages thereof, the following specific Examples are given with the understanding that they are being offered to illustrate the present invention and should not be construed in any way as limiting its scope.

### EXAMPLES

The following Examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); bp (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₄₀₅ (optical density at 405 nm); PAGE (polyacrylamide gel electrophoresis); Tris (tris(hydroxymethyl)aminomethane); MES (2-morpholinoethanesulfonic acid, monohydrate); ppm (parts per million); gpg (grains per gallon of water); HDD (heavy duty detergent); HDL (heavy duty liquid); ADW (automatic dish washing); SRI (Stain Removal Index); AATCC (American Association of Textile and Coloring Chemists); TOT (Terg-o-Tometer); DNA2.0 (DNA2.0, Menlo Park, Calif.); Sigma (Sigma-Aldrich Corp., St. Louis, Mo.); Test Fabrics (Test Fabrics, Inc. West Pittston, Pa.); Corning (Corning Inc., Corning, N.Y.); Pechiney (Pechiney Plastic Packing, Menasha, Wis.); Eppendorf (Eppendorf Scientific, Westbury, N.Y.); VWR (VWR International, West Chester, Pa.); Molecular Devices (Molecular Devices Corp., Sunnyvale, Calif.); Warwick (Warwick Equest Limited, Durham, England); Minolta (Konica Minolta Holdings, Inc., Tokyo, Japan); U.S. Testing (U.S. Testing Co. Inc. Hoboken, N.J.); and Rockland (Rockland Immunochemicals, Gilbertsville, Pa.).

In these experiments, a spectrophotometer was used to measure the absorbance of the products formed after the completion of the reactions. A reflectometer was used to measure the reflectance of the swatches. Unless otherwise indicated, protein concentrations were estimated by Coomassie Plus (Pierce), using BSA as the standard.

### Example 1

### Expression and Secretion of the κ-Carrageenase cgkA Gene in B. subtilis

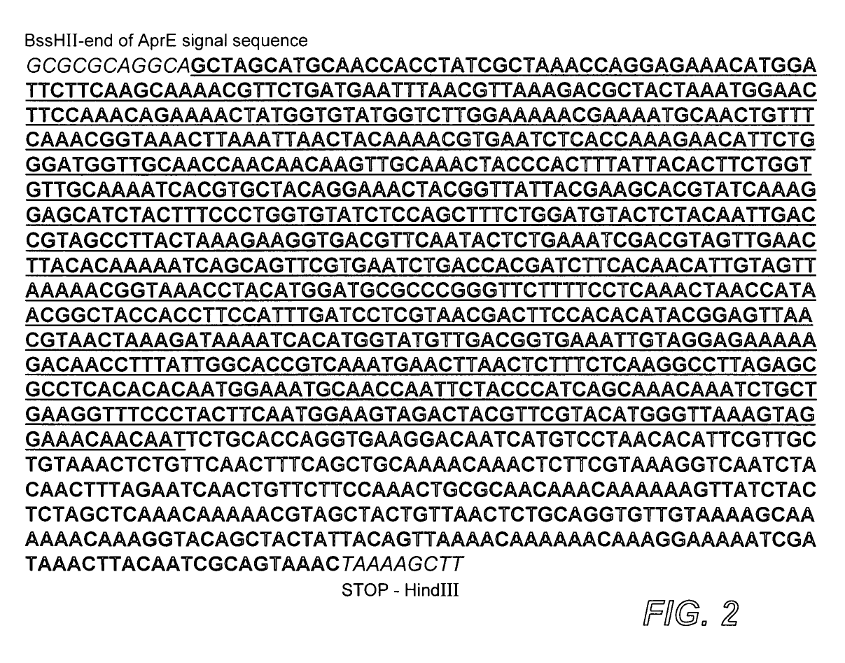

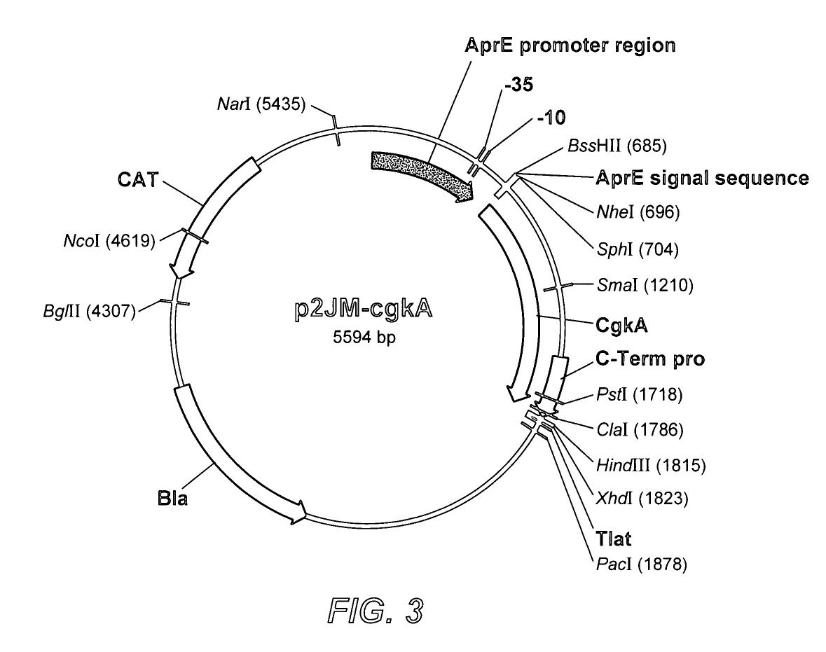

The kappa-carrageenase gene (cgkA gene) (without the signal sequence) from Alteromonas carrageenovora (Potin et al., Eur. J. Biochem., 228:971-975 [1995]) was synthesized with optimal B. subtilis codons (DNA2.0) and cloned in vector pJ31-7585. The codon-optimized gene sequence (SEQ ID NO:1) was ligated into the B. subtilis integration vector p2JM, between the DNA sequence for the aprE promoter followed by the aprE signal sequence, and the LAT terminator) as an 1.1 kb BssHII-HindIII fragment (See, FIG. 2). The resulting vector, p2JM-cgkA (See, FIG. 3), was used to transform B. subtilis host cells, BG3594cK (U.S. Pat. No. 5,387,521), BG3934comK; (US20050202535), and BG6006 (US20050202535) that respectively contain deletions of 2 (nprE and aprE), 5 (nprE, aprE, epr, ispA, and bpr), and 9 (nprE, aprE, epr, ispA, bpr, vpr, wprA, mpr-ybjF and nprB) protease genes. Two clones from the transformation of each host strain were picked and amplified to 25 mg/ml chloramphenicol.

The clones were grown separately in 30 ml Grant's II medium with 25 mg/ml chloramphenicol in 250 ml shake flasks at 37° C. and 250 rpm for ˜60 h. The supernatants from each culture were harvested by centrifugation and analyzed by polyacrylamide gel electrophoresis and for k-carrageenase enzyme activity.

SEQ ID NO: 1

GCTAGCATGCAACCACCTATCGCTAAACCAGGAGAAACATGGATTCTTCA
AGCAAAACGTTCTGATGAATTTAACGTTAAAGACGCTACTAAATGGAACT
TCCAAACAGAAAACTATGGTGTATGGTCTTGGAAAAACGAAAATGCAACT
GTTTCAAACGGTAAACTTAAATTAACTACAAAACGTGAATCTCACCAAAG
AACATTCTGGGATGGTTGCAACCAACAACAAGTTGCAAACTACCCACTTT
ATTACACTTCTGGTGTTGCAAAATCACGTGCTACAGGAAACTACGGTTAT
TACGAAGCACGTATCAAAGGAGCATCTACTTTCCCTGGTGTATCTCCAGC
TTTCTGGATGTACTCTACAATTGACCGTAGCCTTACTAAAGAAGGTGACG
TTCAATACTCTGAAATCGACGTAGTTGAACTTACACAAAAATCAGCAGTT
CGTGAATCTGACCACGATCTTCACAACATTGTAGTTAAAAACGGTAAACC
TACATGGATGCGCCCGGGTTCTTTTCCTCAAACTAACCATAACGGCTACC
ACCTTCCATTTGATCCTCGTAACGACTTCCACACATACGGAGTTAACGTA
ACTAAAGATAAAATCACATGGTATGTTGACGGTGAAATTGTAGGAGAAAA
AGACAACCTTTATTGGCACCGTCAAATGAACTTAACTCTTTCTCAAGGCC
TTAGAGCGCCTCACACACAATGGAAATGCAACCAATTCTACCCATCAGCA
AACAAATCTGCTGAAGGTTTCCCTACTTCAATGGAAGTAGACTACGTTCG
TACATGGGTTAAAGTAGGAAACAACAATTCTGCACCAGGTGAAGGACAAT
CATGTCCTAACACATTCGTTGCTGTAAACTCTGTTCAACTTTCAGCTGCA
AAACAAACTCTTCGTAAAGGTCAATCTACAACTTTAGAATCAACTGTTCT
TCCAAACTGCGCAACAAACAAAAAAGTTATCTACTCTAGCTCAAACAAAA
ACGTAGCTACTGTTAACTCTGCAGGTGTTGTAAAAGCAAAAAACAAAGGT
ACAGCTACTATTACAGTTAAAACAAAAAACAAAGGAAAAATCGATAAACT
TACAATCGCAGTAAAC

Twenty microliters of the carrageenase sample produced by each of the three cultures was analyzed using 10% NuPAGE in MES buffer under reducing conditions. An unrelated protein having a molecular weight of 48.9 kDa, BCE-cAbBCII10, was used as a positive control.

The NuPAGE gel showed that clones A and B of the BG3934cK culture produced the greatest level of k-carrageenase (predicted molecular weight of 31.7 kDa) when compared to the level produced by the clones of the BG3594cK culture. The BG6006 culture did not produce detectable k-carrageenase using this detection method. The supernatant from the BG6006 cultures was used as a negative control in experiments that determined the enzymatic activity of the k-carrageenase produced by the BG3934cK cultures as described in the Examples below.

### Example 2

### Enzymatic Activity and Substrate Specificity of κ-Carrageenase

Hydrolytic activity by enzymes on carrageenan was measured using the reducing sugar assay using the PAHBAH (para-hydroxybenzoic acid hydrazide) reagent (See, Lever, Anal. Biochem., 47:273 [1972]). Carrageenans (Type I kappa-, CAS 9000-07-1, Type II iota-, CAS 9062-07-1, and Type III kappa-, CAS11114-20-8) were purchased from Sigma, and dissolved in 50 mM Hepes buffer pH 7.4 at a concentration of 0.25%. For some experiments AATCC standard heavy duty liquid detergent (AATCC HDL 2003 without brightener (Test Fabrics) was added at 1.5 ml per liter (0.15%). The AATCC HDL liquid detergent contained 12% linear alkyl sulfonates, 8% alcohol ethoxylates, 8% propanediol, 1.2% citric acid, 4% fatty acid and 4% sodium hydroxide with the balance being water. Kappa-carrageenase activity was also tested for its ability to remove kappa-carrageenan type I soil in HDD (1 g/l, pH9.5-10) and ADW (1 g/l, pH10.0-10.5) detergents. The supernatants from each culture producing the kappa-caarrageenase were harvested by centrifugation, and aliquots were analyzed for activity in AATCC HDL. For testing of activity in HDD and ADW, the supernatants were first concentrated by twelvefold.

The assay was performed in a 24 well microplate (COSTAR 3526; Corning) as follows: one ml of buffer (I) was added to well 1 (Buffer), one ml of buffer plus enzyme was added to well 2 (II) (Sample), one ml of buffer and substrate to well 3 (III) (Substrate), and one ml of buffer, plus substrate and enzyme (V) (Enz+Sub) was added to well 4. The bars labeled “Sam+Sub” (IV) reflect the calculated control values. For statistical purposes, each well was set up 2 to 4 times. Enzymes to be tested were usually diluted in reaction buffer from 1 to 10 to 1 to 1000. After all reagents were added, a plastic cover was place over the microplate and the cover and plate intersection was wrapped tightly with several layers of Parafilm (Pechiney) to prevent evaporation. The reaction plate was incubated for 1 to 16 hr, at 37° C. on a shaker rotating at 100 rpm.

Reducing sugar activity was measured using an Eppendorf Mastercycler Gradient thermal cycler and 0.2 ml disposable PCR (polymerase chain reaction) strip tubes and caps (VWR). Reducing sugar reagent was prepared as follows: to 10 ml of 2% sodium hydroxide in distilled water, add 0.15 g of sodium potassium tartrate tetrahydrate (Rochelle Salt; Sigma) and 0.15 g of parahydroxybenzoic acid hydrazide (H-9882; Sigma). This solution, called “PAHBAH reagent” was swirled to solubilize all ingredients and put on ice in the dark until used. This reagent was made fresh daily. Immediately before sample analysis, 0.160 ml of PAHBAH reagent was added to each tube of a PCR strip followed by 5 to 20 ul of enzyme samples and controls. All tubes were capped tightly, placed in the thermal cycler, and incubated for 15 min at 99° C. followed by cooling at 4° C. for at least 15 min. After cooling, strip tube caps were removed and 0.15 ml of each sample was placed in a 96 well flat bottomed microplate (COSTAR 9017; Corning) and read by a Spectra Max 250 Plate Reader (Molecular Devices) at 405 nm against a blank of distilled water.

Each enzyme sample was analyzed as follows: the optical density (OD) of the control sample was subtracted from the OD of the buffer sample and this value was added to the substrate buffer control. The OD of the enzyme plus substrate reaction was compared to the sum of the substrate and sample controls.

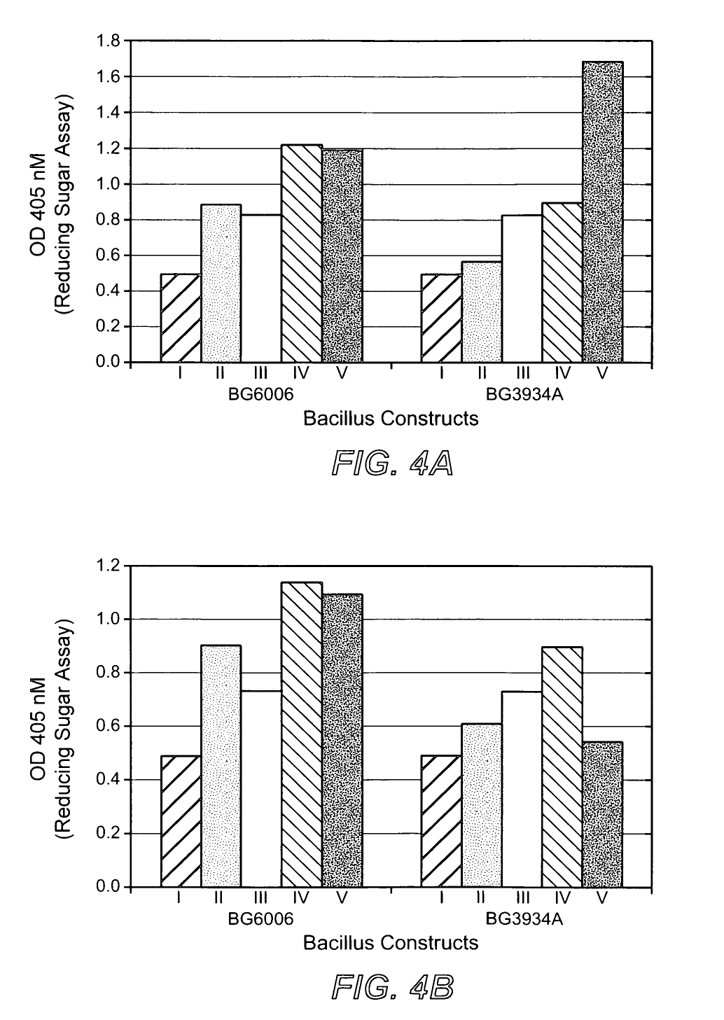

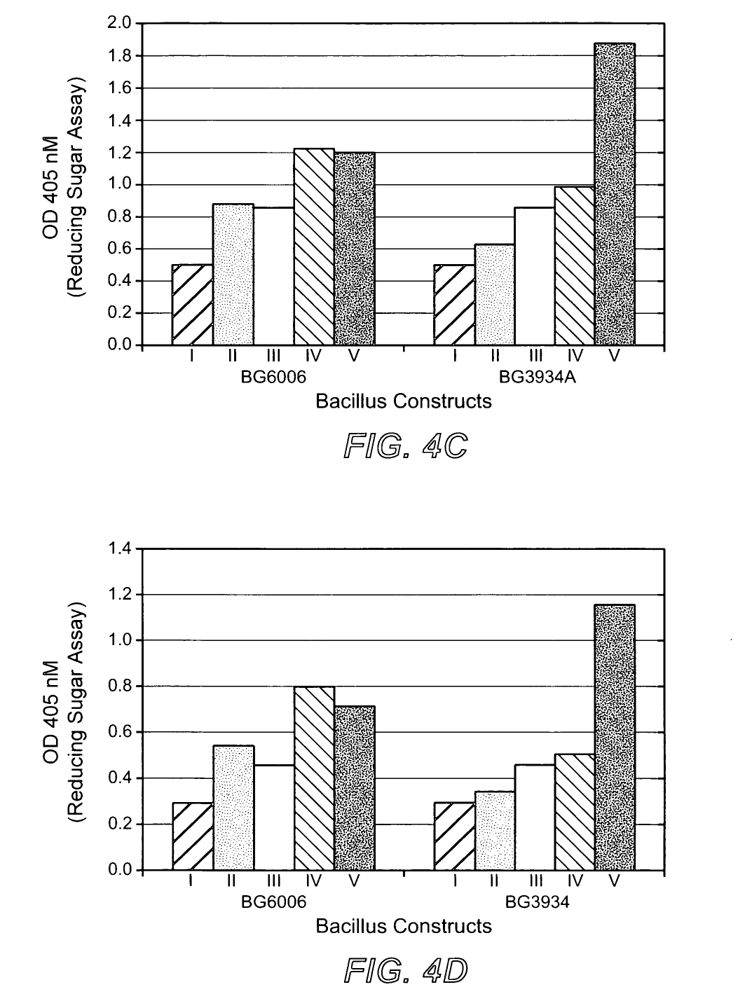

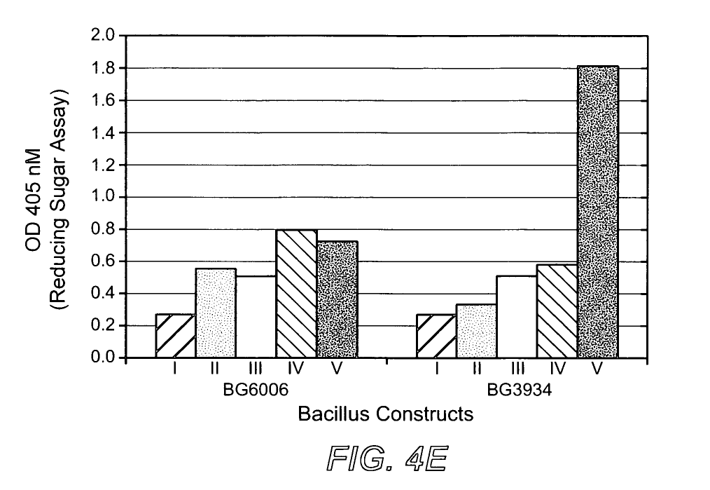

The results showing the hydrolytic activity of the kappa-caargeenase are shown in FIGS. 4A-E. The data show that the kappa-carrageenase effectively removes soil of the type I kappa-carrageenan (FIG. 4A) and the type III kappa carrageenan (FIG. 4C) when used in AATCC HDL liquid detergent.

The results given in FIGS. 4D and 4E show that kappa-carrageenase also removes kappa-carrageenan type I soil when used in HDD (heavy duty detergent) and ADW (Automatic Dish Washing Detergent).

### Example 3

### Cleaning Activity of Cgka Using the Microswatch Screen Assay

The activity of CgkA was tested for its ability to clean swatches stained with salad dressing and barbeque stain using the method described in this Example.

Salad dressing with pigment (STC CFT CS-6) soiled cotton swatches (Test Fabrics) and barbeque stained circles on cotton fabric (Warwick) were used in these experiments.

Swatches for the microplate assay were cut into 15 mm circles (disks) with textile Punch Press Model B equipped with a ⅝″ die cutter. Single swatch disks were placed into each well of a 24-well microplate (Costar 3526). For salad dressing stains, one (1) ml of washing solution, containing per liter, 1.5 ml AATCC HDL, 50 mM Hepes buffer, and 1 to 50 ppm enzyme diluted in 50 mM Hepes buffer pH 7.4, were added to each well. For barbecue stains, one (1 ml) of washing solution containing per liter 1.5 ml of AATCC HDL detergent, 10 mM Hepes pH 8.2, 50 mM sodium chloride, and 2.5 mM calcium chloride were added to each well. Control wells contained no enzyme. The controls for the barbeque stain experiments were AATCC control, and a bovine serum albumin (BSA) control. The microplate was covered with a plastic lid and aluminum foil and incubated at 37° C. with 100 rpm gentle rotation for 3-16 hr. The plates were removed from the shaker and the detergent solution was removed by aspiration. Each microplate well was washed three (3) times with 1.5 ml of Dulbecco's PBS pH 7.3 and three (3) times with 1.5 ml of distilled water. Each disk was removed from its well and dried overnight between sheets of paper towels and not exposed to direct light. Disks were inspected visually and then analyzed with a Minolta Reflectometer CR-200 calibrated on a standard white tile. The average L values with percent standard deviation of the data with generally 4 replicates per control and test sample were calculated. For the experiments measuring the activity of CO-cgkA on barbeque stains, the percent soil release index (% SRI) was calculated from the “L” values according to the following formula: % SRI=L (final)−L (original)/L (white tile)−L (original)×100%.

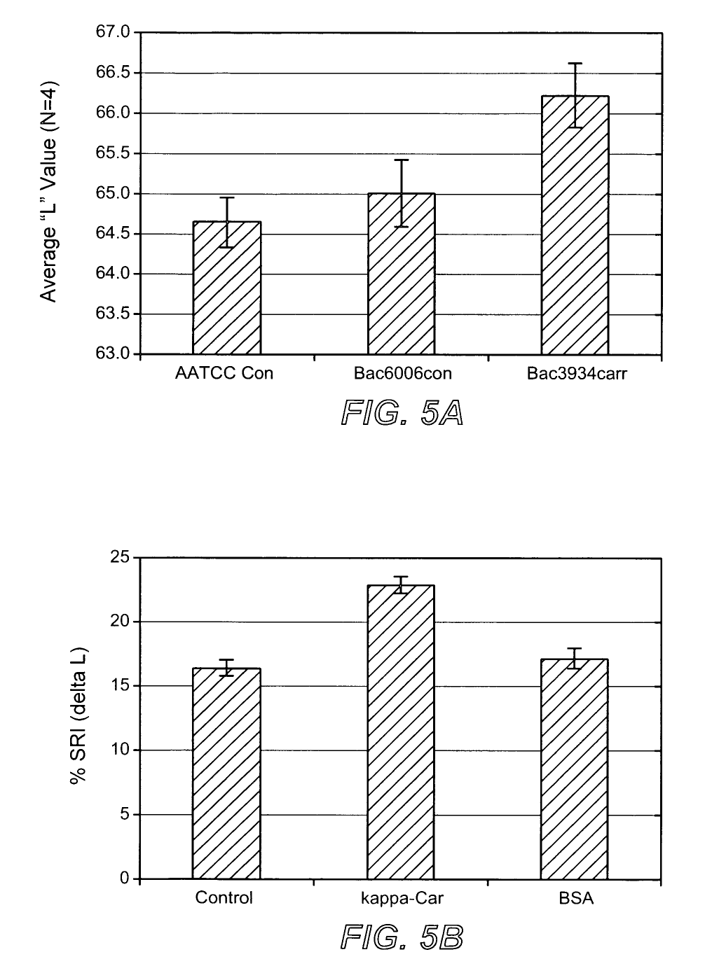

The results of the Microswatch Screen Assays are shown in FIGS. 5A and 5B. As shown in FIGS. 5A and 5B, CgkA showed excellent cleaning on salad dressing and barbeque stains, respectively.

### Example 4

### Cleaning Activity of CgkA Using the Tergotometer

Tergotometer studies used a 6 pot Tergotometer Model 7243S (U.S. Testing) maintained at 30° C. Agitation speed was set to 100 rpm. Cotton swatches (5 per each tergotometer pot) stained with circles of foodstuffs (Warwick) were added to 1 liter of 0.15% AATCC HDL detergent containing 6 gpg hardness (diluted from stock 15000 gpg hardness solution containing 1.735 M calcium chloride and 0.67 M magnesium chloride), and 25 mM Hepes buffer pH 7.4. After a 30 min wash cycle, the swatches were washed three times in 1.5 L of cold tap water, spun for 7 min in a spin cycle to remove excess water, and dried overnight at room temperature. Percent soil release (% SRI) was calculated by standard methods (See, Microswatch Screen Methods) after analysis of each stain by reflectometer. The activity of kappa-carrageenase at 15 ppm (III) relative to a control sample lacking the enzyme (I), was compared to that of a control protein, bovine serum albumin (II) (BSA-50, Fraction V, Immunoglobulin and Protease Free; Rockland).

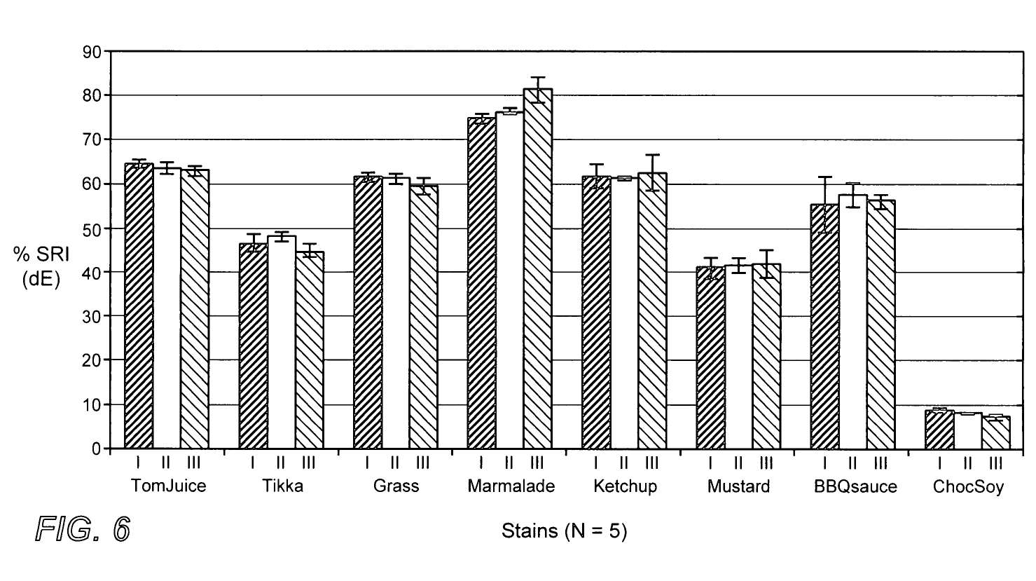

FIG. 6 shows that CgkA has significant cleaning activity in removing marmalade stains in a tergotometer assay. The percent SRI values were calculated from the results obtained for 5 replicate experiments.

The above Examples demonstrate that kappa-carrageenase effectively removes soil from cotton swatches stained with salad dressing, barbeque and marmalade stains.

Having described the preferred embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

We claim:

1. An isolated recombinant polynucleotide comprising a sequence encoding a kappa-carrageenase polypeptide, said sequence being operably linked to a polynucleotide encoding a secretory signal peptide.

2. The isolated polynucleotide of claim 1, wherein said polynucleotide encoding a secretory signal peptide is derived from a Gram-positive microorganism.

3. The isolated polynucleotide of claim 1, wherein said secretory signal peptide is the AprE signal peptide.

4. The isolated polynucleotide of claim 1, wherein said sequence encoding said carrageenase enzyme comprises a sequence that is optimized from a polynucleotide encoding a wild-type carrageenase obtained from Alteromonas sp.

5. The isolated polynucleotide of claim 4, wherein said wild-type carrageenase is derived from Alteromonas carrageenovora.

6. The isolated polynucleotide of claim 4, wherein said optimized polynucleotide is at least about 70% identical to SEQ ID NO:1 or 3.

7. An expression vector comprising an expression cassette comprising the isolated polynucleotide sequence of claim 1.

8. The expression vector of claim 7, wherein said secretory signal peptide is the AprE signal peptide.

9. A host cell transformed with said expression vector of claim 7.

10. A Bacillus host cell comprising:
a recombinant polynucleotide encoding a fusion protein comprising:
a) a signal sequence; and
b) carrageenase protein;
wherein said host cell secretes said kappa-carrageenase protein from said cell and wherein said host cell has at least one inactivated protease gene.

11. The Bacillus host cell of claim 10, wherein said carrageenase protein has an amino acid sequence that is at least about 70% identical to SEQ ID NO:4.

12. The Bacillus host cell of claim 10, wherein said at least one inactivated protease gene is selected from nprE, aprE, epr, ispA, and bpr genes.

13. The Bacillus host cell of claim 10, wherein said inactivated protease genes are nprE and aprE genes.

14. The host cell of claim 10, wherein said signal sequence is the signal sequence encoded by the aprE gene of B. subtilis.

15. The host cell of claim 10, wherein said Bacillus host cell is a B. subtilis host cell.

16. The host cell of claim 10, wherein said recombinant polynucleotide is operably linked to a promoter and terminator to form an expression cassette.

17. The host cell of claim 10, wherein said recombinant polynucleotide is present in the genome of said host cell or in a vector that autonomously replicates in said host cell.

18. The host cell of claim 10, wherein said recombinant polynucleotide is codon optimized for expression of said carrageenase fusion protein in said Bacillus host cell.

19. A culture of cells comprising:
a plurality of Bacillus host cells of claim 10 and
culture medium.

20. A method of producing a carrageenase protein, comprising:
culturing the cell of claim 10 to provide for secretion of said kappa-carrageenase protein in culture medium.

21. The method of claim 21, further comprising the step of harvesting said produced carrageenase.

22. The method of claim 20, wherein said host cell is a Bacillus species.

23. A cleaning composition comprising an effective amount of an isolated kappa-carrageenase comprising an amino acid sequence that is at least about 70% identical to the kappa-carrageenase of SEQ ID NO:4.

24. The cleaning composition of claim 23, wherein said cleaning composition is a detergent.

25. The cleaning composition of claim 24, further comprising at least one additional enzyme or enzyme derivatives.

26. The cleaning composition of claim 25, wherein said at least one additional enzyme or enzyme derivative is selected from hemicellulases, peroxidases, proteases, cellulases, xylanases, lipases, phospholipases, esterases, cutinases, pectinases, keratinases, reductases, oxidases, oxidoreductases, phenoloxidases, lipoxygenases, ligninases, pullulanases, tannases, pentosanases, mannanases, β-glucanases, arabinosidases, hyaluronidases, chondroitinases, laccases, and amylases, or mixtures thereof.

27. A method of cleaning, comprising the step of contacting a hard surface and/or an article comprising a fabric with the cleaning composition of claim 23.

28. The method of claim 27, further comprising the step of rinsing said surface and/or material after contacting said surface or material with said cleaning composition.

29. The method of claim 27, wherein said surface and/or an article comprising a fabric is stained with a kappa-carrageenan.

30. The method of claim 27, wherein said surface and/or said article comprising fabric is soiled with salad dressing, barbeque sauce and/or marmalade.
